(19) United States
(12) Patent Application Publication
(10) Pub. No.: US 2010/0204303 A1
(43) Pub. Date: Aug. 12, 2010

(54) Pharmaceutical Compositions for Administering Oligonucleotides

(75) Inventors: Yerramilli V.S.N. Murthy, Apex, NC (US); Michael Atkinson, Cape Elizabeth, ME (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IDEXX Laboratories, Inc., Westbrook, ME (US)

(21) Appl. No.: 12/678,776

(22) PCT Filed: Sep. 24, 2008
(86) PCT No.: PCT/US08/77426

§ 371 Date: Apr. 20, 2010

Related U.S. Application Data
(60) Provisional application No. 60/960,302, filed on Sep. 25, 2007.

(51) Int. Cl. A61K 31/7088

(52) U.S. Cl. 514/44 A; 514/44 R; 977/773; 977/906

(57) ABSTRACT

The invention relates to pharmaceutical compositions useful for administering an oligonucleotide to an animal in need thereof. The pharmaceutical compositions include nano-particles or micro-particles of (i) a protonated oligonucleotide and (ii) a pharmaceutically acceptable organic base or include nano-particles or micro-particles of (i) an oligonucleotide and (ii) a divalent metal ion.

### 1. CROSS REFERENCE TO RELATED APPLICATIONS

Not Applicable.

### 2. STATEMENT REGARDING FEDERALLY SPONSORED RESEARCH OR DEVELOPMENT

Not Applicable.

### 3. INCORPORATION BY REFERENCE OF MATERIAL SUBMITTED ON A COMPACT DISC

Not Applicable.

### 4. FIELD OF THE INVENTION

The invention relates to pharmaceutical compositions useful for administering an oligonucleotide to an animal in need thereof. In one embodiment, the pharmaceutical composition comprises nano-particles or micro-particles comprising (i) a protonated oligonucleotide and (ii) a pharmaceutically acceptable organic base. In one embodiment, the pharmaceutical composition comprises nano-particles or micro-particles comprising (i) an oligonucleotide and (ii) a divalent metal ion. In one embodiment, the particles are nano-particles. In one embodiment, the particles are micro-particles.

Oligonucleotides are small double-stranded or single-stranded segments of DNA or RNA, typically about 20-30 nucleotide bases in length. Oligonucleotides can be synthetic or natural, and bind to a particular target molecule, such as a protein, metabolite, or other nucleic acid sequence. Oligonucleotides are a promising class of therapeutic agents currently in pre-clinical and clinical development for treating a variety of diseases and disorders. Like biologics, e.g., peptides or monoclonal antibodies, oligonucleotides are capable of binding specifically to molecular targets and, through binding, inhibiting target function. Oligonucleotides include for example, siRNA and aptamers.

SiRNA are small strands of RNA that interfere with the translation of messenger RNA. SiRNA can be double stranded or single stranded. Generally, double stranded siRNA works better than single stranded siRNA. Typically, siRNA are about 20 to 25 nucleotides long. SiRNA can be used to interfere with the expression of genes. They bind to the complementary portion of the target messenger RNA and tag it for degradation. SiRNA's effect of inhibiting gene expression is commonly known as gene “silencing.” The siRNA causes the destruction of messenger RNA that shares sequence homology with the siRNA to within one nucleotide resolution (Elbashir S. M. et al., Genes Dev., 15 (2001) 188-200). It is believed that the siRNA and the targeted mRNA bind to an “RNA-induced silencing complex” or “RISC,” which cleaves the targeted mRNA. The siRNA is apparently recycled much like a multiple-turnover enzyme, with 1 siRNA molecule capable of inducing cleavage of approximately 1000 mRNA molecules. The siRNA mediated degradation of a mRNA is therefore more effective than currently available technologies for inhibiting expression of a target gene.

The ability to specifically inhibit expression of a target gene by siRNA has obvious benefits. For example, many diseases arise from the abnormal expression of a particular gene or group of genes. SiRNA can be used to inhibit the expression of the deleterious gene and therefore alleviate symptoms of a disease or even provide a cure. For example, genes contributing to a cancerous state or to viral replication could be inhibited. In addition, mutant genes causing dominant genetic diseases such as myotonic dystrophy could be inhibited. Inflammatory diseases such as arthritis could also be treated by inhibiting such genes as cyclooxygenase or cytokines. Examples of targeted organs include, but are not limited to the liver, pancreas, spleen, skin, brain, prostrate, heart. In addition, siRNA could be used to generate animals that mimic true genetic “knockout” animals to study gene function. Useful sequences of siRNA can be identified using known procedures such as described in Pharmacogenomics, 6(8):879-83 (December 2005), Nat. Chem. Biol., 2(12):711-9 (December 2006), Appl Biochem. Biotechnol., 119(1):1-12 (October 2004), U.S. Pat. No. 7,056,704 and U.S. Pat. No. 7,078,196).

Aptamers, are oligonucleotides that bind to a particular target molecule, such as a protein or metabolite. Typically, the binding is through interactions other than classic Watson-Crick base pairing. A typical aptamer is 10-15 kDa in size (i.e., 30-45 nucleotides), binds its target with sub-nanomolar affinity, and discriminates among closely related targets (e.g., will typically not bind other proteins from the same gene family) (Griffin, et al. (1993), Gene, 137(1): 25-31; Jenison, et al. (1998), Antisense Nucleic Acid Drug Dev., 8(4): 265-79; Bell, et al. (1999), In Vitro Cell. Dev. Biol. Anim., 35(9): 533-42; Watson, et al. (2000), Antisense Nucleic Acid Drug Dev., 10(2): 63-75; Daniels, et al. (2002), Anal. Biochem., 305(2): 214-26; Chen, et al. (2003), Proc. Natl. Acad. Sci. USA., 100(16): 9226-31; Khati, et al. (2003), J. Virol., 77(23): 12692-8; Vaish, et al. (2003), Biochemistry, 42(29): 8842-51).

Aptamers can be created by an entirely in vitro selection process (Systematic Evaluation of Ligands by Experimental Enrichment, i.e., SELEX™) from libraries of random sequence oligonucleotides as described in U.S. Pat. Nos. 5,475,096 and 5,270,163. Aptamers have been generated against numerous proteins of therapeutic interest, including growth factors, enzymes, immunoglobulins, and receptors (Ellington and Szostak (1990), Nature, 346(6287): 818-22; Tuerk and Gold (1990), Science, 249(4968): 505-510).

Aptamers have a number of attractive characteristics for use as therapeutics. In addition to high target affinity and specificity, aptamers have shown little or no toxicity or immunogenicity in standard assays (Wlotzka, et al. (2002), Proc. Natl. Acad. Sci. USA., 99(13): 8898-902). Indeed, several therapeutic aptamers have been optimized and advanced through varying stages of pre-clinical development, including pharmacokinetic analysis, characterization of biological efficacy in cellular and animal disease models, and preliminary safety pharmacology assessment (Reyderman and Stavchansky (1998), Pharmaceutical Research, 15(6): 904-10; Tucker et al., (1999), J. Chromatography B., 732: 203-212; Watson, et al. (2000), Antisense Nucleic Acid Drug Dev., 10(2): 63-75).

Oligonucleotides, to be effective, must be distributed to target organs and tissues, and remain in the body (unmodified) for a period of time consistent with the desired dosing regimen. In addition, siRNA, to be effective, must enter the cell. Aptamers, however, are directed against extracellular targets and, therefore, do not suffer from difficulties associated with intracellular delivery.

It is important, however, that the pharmacokinetic properties for all oligonucleotide-based therapeutics be tailored to match the desired pharmaceutical application. Early work on nucleic acid-based therapeutics has shown that, while unmodified oligonucleotides are degraded rapidly by nuclease digestion, protective modifications at the 2′-position of the sugar, and use of inverted terminal cap structures, e.g., [3′-3′ dT], dramatically improve nucleic acid stability in vitro and in vivo (Green, et al. (1995), Chem. Biol., 2(10): 683-95; Jellinek, et al. (1995), Biochemistry, 34(36): 11363-72; Rudman, et al. (1998), J. Biol. Chem., 273(32): 20556-67; Uhlmann, et al. (2000), Methods Enzymol., 313: 268-84). For example, in some SELEX selections (i.e., SELEX experiments or SELEX ions), the starting pools of nucleic acids from which aptamers are selected are typically pre-stabilized by chemical modification, for example by incorporation of 2′-fluoropyrimidine (2′-F) substituted nucleotides, to enhance resistance of the aptamers against nuclease attack. Aptamers incorporating 2′-O-methylpurine (2′-OMe purine) substituted nucleotides have also been developed through post-SELEX modification steps or, more recently, by enabling synthesis of 2′-OMe-containing random sequence libraries as an integral component of the SELEX process itself.

In addition to clearance by nucleases, oligonucleotide therapeutics are subject to elimination via renal filtration. As such, a nuclease-resistant oligonucleotide administered intravenously exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation to a PEG polymer (“PEGylation”) can dramatically lengthen residence times of oligonucleotides in circulation, thereby decreasing dosing frequency and enhancing effectiveness against targets. Previous work in animals has examined the plasma pharmacokinetic properties of PEG-conjugated aptamers (Reyderman and Stavchansky (1998), Pharmaceutical Research, 15(6): 904-10; Watson, et al. (2000), Antisense Nucleic Acid Drug Dev., 10(2): 63-75)). Determining the extravasation of an oligonucleotide therapeutic, including oligonucleotide therapeutics conjugated to a modifying moiety or containing modified nucleotides and, in particular, determining the potential of oligonucleotides or their modified forms to access diseased tissues (for example, sites of inflammation, or the interior of tumors) define the spectrum of therapeutic opportunities for oligonucleotide intervention.

Typically, therapeutic oligonucleotides are administered by injection, for example, by subcutaneous or intravenous injection. Accordingly, the oligonucleotides must be dissolved or dispersed in a liquid vehicle for administration. The relatively high molecular weight of oligonucleotides, and in particular oligonucleotides that have been derivatized, for example by PEGylation, however, often makes it difficult to obtain a pharmaceutical composition wherein the oligonucleotide is dissolved or dispersed in a pharmaceutically acceptable solvent at a sufficient concentration to provide a pharmaceutical composition that is clinically useful for administration to an animal.

U.S. published application no. 2005/0175708 discloses a composition of matter that permits the sustained delivery of aptamers to a mammal. The aptamers are administered as microspheres that permit sustained release of the aptamers to the site of interest so that the aptamers can exert their biological activity over a prolonged period of time. The aptamers, can be anti-VEGF aptamers.

P. Burmeister et al., (2004), Chemistry and Biology: 15, 25-33 disclose a method for generating a 2′-O-methyl aptamer (ARC245) that binds to vascular endothelial growth factor, which exhibits good stability.

There is a need in the art for improved pharmaceutical compositions, wherein the therapeutic agent is an oligonucleotide. In particular, there is a need for pharmaceutical composition wherein the oligonucleotide can be dissolved or dispersed in a pharmaceutically acceptable solvent at a sufficient concentration to provide a pharmaceutical composition that is clinically useful for administration to an animal, and, in particular, administration by injection. The present invention addresses this as well as other needs.

Citation of any reference in this application is not to be construed as an admission that such reference is prior art to the present application.

### 5. SUMMARY OF THE INVENTION

The invention is directed to a pharmaceutical composition comprising nano-particles comprising: (i) a protonated oligonucleotide and (ii) a pharmaceutically acceptable organic base.

The invention is further directed to a pharmaceutical composition comprising micro-particles comprising: (i) a protonated oligonucleotide and (ii) a pharmaceutically acceptable organic base.

In one embodiment, the pharmaceutically acceptable organic base is an amino acid ester.

In one embodiment, the pharmaceutically acceptable organic base is an amino acid amide.

In one embodiment, the pharmaceutically acceptable organic base is an amino acid vitamin ester.

The invention is further directed to a pharmaceutical composition comprising nano-particles comprising (i) an oligonucleotide (ii) a divalent metal cation; and (iii) optionally a carboxylic acid, a phospholipid, a phosphatidyl choline, or a sphingomyelin.

The invention is further directed to a pharmaceutical composition comprising micro-particles comprising (i) an oligonucleotide (ii) a divalent metal cation; and (iii) optionally a carboxylic acid, a phospholipid, a phosphatidyl choline, or a sphingomyelin.

The invention further relates to a method of administering an oligonucleotide to an animal comprising administering to the animal a composition of the invention. In one embodiment, the nano-particles or micro-particles are dispersed in a solvent and the administering is by injection.

The invention is further directed to methods of treating or preventing a condition in an animal comprising administering to the animal a pharmaceutical composition of the invention. In one embodiment, the nano-particles or micro-particles are dispersed in a solvent and the administering is by injection.

### 6. BRIEF DESCRIPTION OF THE DRAWINGS

FIG. 1 is an electron microscope image of particles prepared as described in Example 2.

### 7. DETAILED DESCRIPTION OF THE INVENTION

### 7.1 Definitions

As used herein, the following terms have the following meaning:

The term “oligonucleotide,” as used herein, means small double-stranded or single-stranded segments of DNA or RNA, typically about 5-50 nucleotides in length. In one embodiment, the oligonucleotide is about 5-45 nucleotide bases in length. In one embodiment, the oligonucleotide is about 5-30 nucleotide bases in length. In one embodiment, the oligonucleotide is about 10-50 nucleotide bases in length. In one embodiment, the oligonucleotide is about 10-45 nucleotide bases in length. In one embodiment, the oligonucleotide is about 10-30 nucleotide bases in length. In one embodiment, the oligonucleotide is about 20-50 nucleotide bases in length. In one embodiment, the oligonucleotide is about 20-45 nucleotide bases in length. In one embodiment, the oligonucleotide is about 20-30 nucleotide bases in length. The term “protonated oligonucleotide,” as used herein, means an oligonucleotide wherein at least one of the phosphate groups of the oligonucleotide is protonated. In one embodiment, all of the phosphate groups of the oligonucleotide are protonated.

The term “aptamer,” as used herein, means an oligonucleotide, which can be synthetic or natural, which can bind to a particular target molecule, such as a protein or metabolite, other than by Watson-Crick base pairing and have a pharmacological effect in an animal. Aptamers can be synthesized using conventional phosphodiester linked nucleotides and synthesized using standard solid or solution phase synthesis techniques which are known to those skilled in the art (See, for example, U.S. Pat. Nos. 5,475,096 and 5,270,163). The binding of aptamers to a target polypeptide can be readily tested by assays known to those skilled in the art (See, Burmeister et al., Chem. Biol., 12: 25-33 (2005), U.S. Pat. No. 5,270,163, and U.S. Pat. No. 5,595,877). The term “protonated aptamer,” as used herein, means an aptamer wherein at least one of the phosphate groups of the aptamer is protonated. In one embodiment, all of the phosphate groups of the aptamer are protonated.

The term “siRNA,” as used herein means an oligonucleotide, which can be synthetic or natural, which can bind to another nucleotide sequence, such as that of messenger RNA, by Watson-Crick base pairing and have a pharmacological effect in an animal. SiRNA can also be synthesized using conventional phosphodiester linked nucleotides and synthesized using standard solid or solution phase synthesis techniques which are known to those skilled in the art (See, for example, U.S. Pat. Nos. 7,056,704 and 7,078,196). The identification of siRNA that will bind to a target nucleic acid sequence can be readily determined by methods known to those skilled in the art (See, for example, Pharmacogenomics, 6(8):879-83 (December 2005), Nat. Chem. Biol., 2(12):711-9 (December 2006), Appl Biochem. Biotechnol., 119(1):1-12 (October 2004)). The term “protonated siRNA,” as used herein, means siRNA wherein at least one of the phosphate groups of the siRNA is protonated. In one embodiment, all of the phosphate groups of the siRNA are protonated.

The term “antisense nucleic acid,” as that term is used herein, means a non-enzymatic nucleic acid molecule that binds to target RNA by means of RNA-RNA or RNA-DNA or RNA-PNA (protein nucleic acid; Egholm et al., Nature, 365 (1993) 566) interactions and alters the activity of the target RNA (for a review, see, Stein and Cheng, Science, 261 (1993) 1004 and U.S. Pat. No. 5,849,902). For a review of current antisense strategies, see, Schmajuk et al., J. Biol. Chem., 274 (1999) 21783-21789, Delihas et al., Nature, 15 (1997) 751-753, Stein et al., Antisense N. A. Drug Dev., 7 (1997) 151, Crooke, Methods Enzymol., 313 (2000) 3-45; Crooke, Biotech. Genet. Eng. Rev., 15 (1998) 121-157, and Crooke, Ad. Pharmacol, 40 (1997) 1-49). The identification of an antisense nucleic acid that will bind to a target nucleic acid sequence can be readily determined by methods known to those skilled in the art (See, for example, U.S. Pat. No. 5,639,595, U.S. Pat. No. 5,686,242, N. M. Dean, Functional genomics and target validation approaches using antisense oligonucleotides technology, Curr. Opin. Biotechnol., 12(6):622-5 (2001), R. S. Geary et al., Pharmacokinetics of phosphorothioate antisense oligodeoxynucleotides. Curr. Opin. Investig. Drugs, 2(4):562-573 (2001), S. T. Crooke, Progress in antisense technology: The end of the beginning, Methods Enzymol., 313 (Antisense Technology, Part A):3-45 (2000), and S. T. Crooke, Antisense Therapeutics, Biotechnol Genet Eng Rev. 15:121-57 (1998). The term “protonated antisense nucleic acid,” as used herein, means an antisense nucleic acid wherein at least one of the phosphate groups of the antisense nucleic acid is protonated. In one embodiment, all of the phosphate groups of the antisense nucleic acid are protonated.

Typically, the pharmacological effect is treating or preventing a condition in an animal.

The term “condition,” as used herein means an interruption, cessation, or disorder of a bodily function, system, or organ. Representative conditions include, but are not limited to, diseases such as cancer, inflammation, diabetes, and organ failure.

The phrase “treating,” “treatment of,” and the like includes the amelioration or cessation of a specified condition.

The phrase “preventing,” “prevention of,” and the like include the avoidance of the onset of a condition.

The term “nano-particles,” as used herein means particles having an average particle size less than about 250 nm. In one embodiment, the “nano-particles” have an average particle size less than about 200 nm. In one embodiment, the “nano-particles” have an average particle size less than about 180 nm. In one embodiment, the “nano-particles” have an average particle size less than about 160 nm. In one embodiment, the “nano-particles” have an average particle size between about 80 nm and 250 nm. In one embodiment, the “nano-particles” have an average particle size between about 80 nm and 200 nm. In one embodiment, the “nano-particles” have an average particle size between about 80 nm and 180 nm. In one embodiment, the “nano-particles” have an average particle size between about 80 nm and 160 nm. Particle size can be determined using methods well known to those skilled in the art (See, for example, Advanced Drug Delivery Reviews, 47:165-196 (2001), Biomaterials, 24:1781-1785 (2003), and Gene Therapy, 13:646-651 (2006)).

The term “micro-particles,” as used herein means particles having an average particle size less than about 5 μm. In one embodiment, the “micro-particles” have an average particle size less than about 4 μm. In one embodiment, the “micro-particles” have an average particle size less than about 3 μm. In one embodiment, the “micro-particles” have an average particle size less than about 2 μm. In one embodiment, the “micro-particles” have an average particle size less than about 1 μm. In one embodiment, the “micro-particles” have an average particle size greater than about 0.5 μm. In one embodiment, the “micro-particles” have an average particle size between about 0.2 μm and about 5 μm. In one embodiment, the “micro-particles” have an average particle size between about 0.5 μm and about 5 μm. In one embodiment, the “micro-particles” have an average particle size between about 1 μm and about 5 μm. Particle size can be determined using methods well known to those skilled in the art (See, for example, Advanced Drug Delivery Reviews, 47:165-196 (2001), Biomaterials, 24:1781-1785 (2003), and Gene Therapy, 13:646-651 (2006)).

“C₁-C₂₂ hydrocarbon group” means a straight or branched, saturated or unsaturated, cyclic or non-cyclic, aromatic or non-aromatic, carbocyclic or heterocyclic group having from 1 to 22 carbon atoms. Similarly, phrases such as “C₁-C₂₂ hydrocarbon group,” “C₁-C₁₆ hydrocarbon group,” “C₁-C₁₀ hydrocarbon group,” “C₁-C₅ hydrocarbon group,” “C₁-C₃ hydrocarbon group,” “C₁₆-C₂₂ hydrocarbon group,” “C₈-C₁₈ hydrocarbon group,” “C₁₀-C₁₈ hydrocarbon group,” and “C₁₆-C₁₈ hydrocarbon group” means a straight or branched, saturated or unsaturated, cyclic or non-cyclic, aromatic or non-aromatic, carbocyclic or heterocyclic group having from 1 to 21 carbon atoms, from 1 to 16 carbon atoms, from 1 to 10 carbon atoms, from 1 to 5 carbon atoms, 1 to 3 carbon atoms, 16 to 22 carbon atoms, 8 to 18 carbon atoms, 10 to 18 carbon atoms, and 16 to 18 carbon atoms, respectively. Accordingly, the phrase “an acyl group of formula —C(O)—R₁, wherein R₁ is a C₁ to C₂₁ group means an acyl group of formula —C(O)—R₁, wherein R₁ is a straight or branched, saturated or unsaturated, cyclic or non-cyclic, aromatic or non-aromatic, carbocyclic or heterocyclic hydrocarbon group having from 1 to 21 carbon atoms. Representative acyl groups of formula —C(O)—R₁, wherein R₁ is an unsubstituted C₁ to C₂₁ group include, but are not limited to, acetyl, propionyl, butanoyl, hexanoyl, caproyl, laurolyl, myristoyl, palmitoyl, stearoyl, palmioleoyl, oleoyl, linoleoyl, linolenoyl, and benzoyl.

The term “lower alkyl,” as used herein means a C₁-C₆ hydrocarbon group.

The term “salt,” as used herein, means two compounds that are not covalently bound but are chemically bound by ionic interactions.

The term “pharmaceutically acceptable,” as used herein, when referring to a component of a pharmaceutical composition means that the component, when administered to an animal, does not have undue adverse effects such as excessive toxicity, irritation, or allergic response commensurate with a reasonable benefit/risk ratio. Accordingly, the term “pharmaceutically acceptable organic solvent,” as used herein, means an organic solvent that when administered to an animal does not have undue adverse effects such as excessive toxicity, irritation, or allergic response commensurate with a reasonable benefit/risk ratio. Preferably, the pharmaceutically acceptable organic solvent is a solvent that is generally recognized as safe (“GRAS”) by the United States Food and Drug Administration (“FDA”). Similarly, the term “pharmaceutically acceptable organic base,” as used herein, means an organic base that when administered to an animal does not have undue adverse effects such as excessive toxicity, irritation, or allergic response commensurate with a reasonable benefit/risk ratio.

The term “fatty acid,” as used herein means a carboxylic acid of formula R—C(O)OH, wherein R a is C₆-C₂₂ linear or branched, saturated or unsaturated, hydrocarbon group. Representative fatty acids include, but are not limited to, caproic acid, lauric acid, myristic acid, palmitic acid, stearic acid, palmic acid, oleic acid, linoleic acid, and linolenic acid.

The term “polycarboxylic acid,” as that term is used herein means a polymeric compound having more than one —C(O)OH group. One of ordinary skill in the art would readily recognize polymeric compounds that have more than one —C(O)OH group. Representative polycarboxylic acids include, but are not limited to, hyaluronic acid, polyglutamic acid, polyaspartic acid, and polyacrylic acid.

The phrase “injectable” or “injectable composition,” as used herein, means a composition that can be drawn into a syringe and injected intravenously, subcutaneously, intraperitoneally, or intramuscularly into an animal without causing adverse effects due to the presence of solid material in the composition. Solid materials include, but are not limited to, crystals, gummy masses, and gels. Typically, an “injectable composition” can be drawn into an 18 gauge syringe and injected intravenously, subcutaneously, intraperitoneally, or intramuscularly into an animal without causing adverse effects due to the presence of solid material in the composition.

The term “solution,” as used herein, means a uniformly dispersed mixture at the molecular or ionic level of one or more substances (solute), in one or more other substances (solvent), typically a liquid.

The term “suspension” or “dispersion,” as used herein, means solid particles that are evenly dispersed in a solvent, which can be aqueous or non-aqueous. Dispersions can be distinguished from solutions using methods well known to those skilled in the art, for example, using a particle size analyzer such as is commercially available from Malvern Instruments of Worcestershire, England.

The term “animal,” as used herein, includes, but is not limited to, humans, canines, felines, equines, bovines, ovines, porcines, amphibians, reptiles, and avians. Representative animals include, but are not limited to a cow, a horse, a sheep, a pig, an ungulate, a chimpanzee, a monkey, a baboon, a chicken, a turkey, a mouse, a rabbit, a rat, a guinea pig, a dog, a cat, and a human. In one embodiment, the animal is a mammal. In one embodiment, the animal is a human. In one embodiment, the animal is a non-human. In one embodiment, the animal is a canine, a feline, an equine, a bovine, an ovine, or a porcine.

The term “effective amount,” as used herein, means an amount sufficient to treat or prevent a condition in an animal.

The term “phospholipid,” as used herein, means a compound having the general formula:

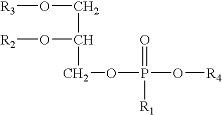

wherein

R₁ is O⁻ or —OH;

R₂ is:
(i) —H, or
(ii) a C₂-C₃₆ saturated or unsaturated, linear or branched acyl group;

R₃ is:
(i) —H,
(ii) a C₂-C₃₆ saturated or unsaturated, linear or branched acyl group; or
(iii) —C═C—R₉ wherein R₉ is a C₁-C₂₂ saturated or unsaturated, linear or branched hydrocarbon group, optionally substituted with one or more nitrogen containing groups;

and at least one of R₂ or R₃ is not —H;

R₄ is:
(i) —H;
(ii) —(CH₂)ₙ—R₅,
   wherein R₅ is —N(R₆)(R₇) or —N(R₆)(R₇) or —N⁺(R₆)(R₇)(R₈),
   R₆, R₇, and R₈ are each independently —H, C₁-C₃ alkyl group, or R₆ and R₇ are connected to form a 5- or 6-membered heterocyclic ring with the nitrogen, and
   n is an integer ranging from 1 to 4, preferably 2;

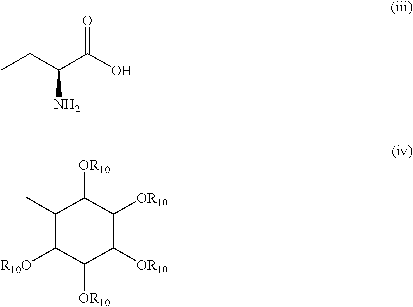

wherein each R₁₀ is independently —H or —P(O)(OH)₂; or
(v) —CH₂CH(OH)CH₂(OH).

The term “saturated or unsaturated, linear or branched C₂-C₃₆ acyl group,” as used herein, means a group of formula —O—C(O)—R, wherein R is a C₁-C₃₅ hydrocarbon group that can be saturated or unsaturated, linear or branched.

The term “sphingomyelin,” as used herein, means a compound having the general formula:

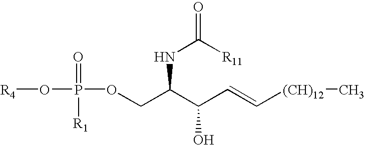

wherein

R₁ is O⁻ or —OH;

R₄ is:
(i) —H; or
(ii) —(CH₂)ₙ—R₅,
   wherein R₅ is —N(R₆)(R₇) or —N⁺(R₆)(R₇)(R₈),
   R₆, R₇, and R₈ are each independently —H, C₁-C₃ alkyl, or R₆ and R₇ are connected to form a 5- or 6-membered heterocyclic ring with the nitrogen, and
   n is an integer ranging from 1 to 4, preferably 2; and
   R₁₁ is a C₁-C₂₂ saturated or unsaturated, linear or branched hydrocarbon group optionally substituted with one or more nitrogen containing groups.

The term “vitamin,” as used herein, is its art recognized meaning, i.e., nutrients required in tiny amounts for essential metabolic reactions in the body. The term vitamin, however, does not include other essential nutrients such as dietary minerals, essential fatty acids, or essential amino acids, nor does it encompass the large number of other nutrients that promote health but that are not essential for life.

The phrase “residue of a vitamin,” as used herein, means a vitamin that has a hydroxyl (i.e., —OH group) wherein the hydrogen of the hydroxyl group is removed. For example, if the formula of the vitamin is H—O—R₁, the formula for the “residue of the vitamin” will be —OR₁.

The term “about,” as used herein to describe a range of values, applies to both the upper limit and the lower limit of the range. For example, the phrase “ranges from about 90:10 to 10:90” has the same meaning as “ranges from about 90:10 to about 10:90.”

### 7.2 The Oligonucleotide

The oligonucleotide can be any oligonucleotide known to those skilled in the art.

In one embodiment, the oligonucleotide is a DNA strand. In one embodiment, the DNA is double stranded DNA. In one embodiment, the DNA is single stranded DNA.

In one embodiment, the oligonucleotide is an RNA strand.

In one embodiment, the oligonucleotide is an aptamer.

In one embodiment, the oligonucleotide is an siRNA.

In one embodiment, the oligonucleotide is an antisense nucleic acid.

In one embodiment, the oligonucleotide has a molecular weight of up to 80 kD. In one embodiment, the molecular weight of the oligonucleotide ranges from about 15 kD to 80 kD. In one embodiment, the molecular weight of the oligonucleotide ranges from about 10 kD to 80 kD. In one embodiment, the molecular weight of the oligonucleotide ranges from about 5 kD to 80 kD.

In one embodiment, the oligonucleotide has a molecular weight of up to 60 kD. In one embodiment, the molecular weight of the oligonucleotide ranges from about 15 kD to 60 kD. In one embodiment, the molecular weight of the oligonucleotide ranges from about 10 kD to 60 kD. In one embodiment, the molecular weight of the oligonucleotide ranges from about 5 kD to 60 kD.

In one embodiment, the oligonucleotide has a molecular weight of up to 40 kD. In one embodiment, the molecular weight of the oligonucleotide ranges from about 15 kD to 40 kD. In one embodiment, the molecular weight of the oligonucleotide ranges from about 10 kD to 40 kD. In one embodiment, the molecular weight of the oligonucleotide ranges from about 5 kD to 40 kD.

In one embodiment, the oligonucleotide has a molecular weight of up to 30 kD. In one embodiment, the molecular weight of the oligonucleotide ranges from about 15 kD to 30 kD. In one embodiment, the molecular weight of the oligonucleotide ranges from about 10 kD to 30 kD. In one embodiment, the molecular weight of the oligonucleotide ranges from about 5 kD to 30 kD.

In one embodiment, the oligonucleotide has a molecular weight of more than 20 kD. In one embodiment, the molecular weight of the oligonucleotide ranges from about 10 kD to 20 kD. In one embodiment, the molecular weight of the oligonucleotide ranges from about 5 kD to 20 kD.

In one embodiment, the molecular weight of the oligonucleotide ranges from about 5 kD to 10 kD.

The nucleotides that make up the oligonucleotide can be modified to, for example, improve their stability, i.e., improve their in vivo half-life, and/or to reduce their rate of excretion when administered to an animal. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or 2′-azido-ribose; carbocyclic sugar analogues; α-anomeric sugars; and epimeric sugars such as arabinose, xyloses or lyxoses, pyranose sugars, furanose sugars, and sedoheptulose.

Modified nucleotides are known in the art and include, but are not limited to, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; and 1-methylcytosine.

The oligonucleotide can also be modified by replacing one or more phosphodiester linkages with alternative linking groups. Alternative linking groups include, but are not limited to embodiments wherein P(O)O is replaced by P(O)S, P(S)S, P(O)NR₂, P(O)R, P(O)OR′, CO, or CH₂, wherein each R or R′ is independently H or a substituted or unsubstituted C₁-C₂₀ alkyl. A preferred set of R substitutions for the P(O)NR₂ group are hydrogen and methoxyethyl. Linking groups are typically attached to each adjacent nucleotide through an —O— bond, but may be modified to include —N— or —S— bonds. Not all linkages in an oligomer need to be identical.

The oligonucleotide can also be modified by conjugating the oligonucleotide to a polymer, for example, to reduce the rate of excretion when administered to an animal. For example, the oligonucleotide can be “PEGylated,” i.e., conjugated to polyethylene glycol (“PEG”). In one embodiment, the PEG has an average molecular weight ranging from about 20 kD to 80 kD. Methods to conjugate an oligonucleotide, specifically an aptamer, with a polymer, such PEG, are well known to those skilled in the art (See, e.g., Greg T. Hermanson, Bioconjugate Techniques, Academic Press, 1966)

In one embodiment, the oligonucleotide is conjugated to a polymer.

In one embodiment, the oligonucleotide is an RNA strand that has been conjugated to a polymer.

In one embodiment, the oligonucleotide is an DNA strand that has been conjugated to a polymer.

In one embodiment, the oligonucleotide is conjugated to PEG.

In one embodiment, the oligonucleotide is an RNA strand that has been conjugated to PEG.

In one embodiment, the oligonucleotide is an DNA strand that has been conjugated to PEG.

In one embodiment, the oligonucleotide is a RNA strand wherein at least one of the 2′-hydroxyls on the sugars that make up the oligonucleotide are O-methylated.

In one embodiment, the oligonucleotide is a RNA strand wherein at least one of the 2′-hydroxyls on the sugars that make up the oligonucleotide are O-methylated and wherein the RNA strand has been conjugated to a polymer.

In one embodiment, the oligonucleotide is a RNA strand wherein at least one of the 2′-hydroxyls on the nucleotides that make up the oligonucleotide are O-methylated and wherein the RNA strand has been conjugated to PEG.

In one embodiment, the oligonucleotide is an aptamer that binds to VEGF (vascular endothelial growth factor).

As an example of a modified aptamer useful in the compositions and methods of the invention see P. Burmeister et al., Direct In Vitro Selection of a 2′-O-methyl Aptamer to VEGF, Chemistry and Biology, vol. 12, 25-33, January 2005.

In one embodiment, the aptamer is ARC224 identified in P. Burmeister et al., Direct In Vitro Selection of a 2′-O-methyl Aptamer to VEGF, Chemistry and Biology, vol. 12, 25-33, January 2005.

In one embodiment, the aptamer is ARC245 identified in P. Burmeister et al., Direct In Vitro Selection of a 2′-O-methyl Aptamer to VEGF, Chemistry and Biology, vol. 12, 25-33, January 2005.

In one embodiment, the aptamer is ARC225 identified in P. Burmeister et al., Direct In Vitro Selection of a 2′-O-methyl Aptamer to VEGF, Chemistry and Biology, vol. 12, 25-33, January 2005.

In one embodiment, the aptamer is ARC259 identified in P. Burmeister et al., Direct In Vitro Selection of a 2′-O-methyl Aptamer to VEGF, Chemistry and Biology, vol. 12, 25-33, January 2005.

In one embodiment, the aptamer is ARC259 identified in P. Burmeister et al., Direct In Vitro Selection of a 2′-O-methyl Aptamer to VEGF, Chemistry and Biology, vol. 12, 25-33, January 2005 wherein the 5′ phosphate group of the aptamer has been pegylated with:

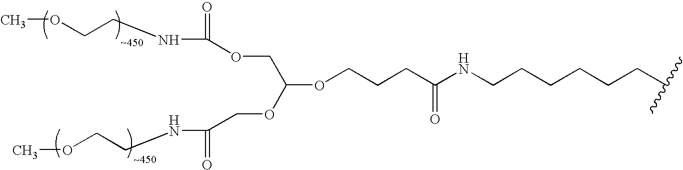

(referred to hereinafter as “pegylated ARC259”).

### 7.3 The Organic Base

Any organic base known to those of ordinary skill in the art can be used in the pharmaceutical compositions of the invention. Preferably, the organic base is a pharmaceutically acceptable organic base. Representative organic bases include, but are not limited to, organic amines including, but not limited to, ammonia; unsubstituted or hydroxy-substituted mono-, di-, or tri-alkylamines such as cyclohexylamine, cyclopentylamine, cyclohexylamine, dicyclohexylamine; tributyl amine, N-methylamine, N-ethylamine, diethylamine, dimethylamine, triethylamine, mono-, bis-, or tris-(2-hydroxy-lower alkyl amines) (such as mono-, bis-, or tris-(2-hydroxyethyl)amine, 2-hydroxy-tert-butylamine, and tris-(hydroxymethyl)methylamine), N,N,-di-lower alkyl-N-(hydroxy lower alkyl)-amines (such as N,N,-dimethyl-N-(2-hydroxyethyl)amine or N,N-dialkyl-N-tris-(2-hydroxyethyl)amines)); pyridine; benzylamine; phenethylamine; N-methyl-D-glucamine; N,N′-dibenzylethylenediamine; chloroprocaine; choline; procaine, and amino acids such as arginine, lysine (See, also, Berge et al., J. Pharm. 1977, 66, 1).

In one embodiment, the amine is an amino acid ester.

In one embodiment, the amine is an amino acid amide.

In one embodiment, the amine is an amino acid-vitamin ester.

In one embodiment, the amine is a diamine (for example, N,N′-dibenzylethylenediamine or an ester or amide of lysine).

In one embodiment, the amine is a diamine and the pharmaceutical composition further comprises a carboxylic acid, a phospholipid, a sphingomyelin, or phosphatidyl choline.

### 7.3.1 The Amino Acid Ester

The amino acid ester can be any ester of any amino acid, i.e., an amino acid wherein the carboxylic acid group of the amino acid is esterified with a C₁-C₂₂ alcohol. Accordingly, the amino acid esters have the general formula (I):

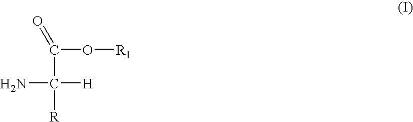

wherein

R is the amino acid side chain; and

R₁ is a C₁ to C₂₂ hydrocarbon group.

As one of ordinary skill in the art would readily know, a wide variety of groups are possible for the amino acid side, R. For example, the amino acid side can be a hydrocarbon group that can be optionally substituted. Suitable substituents include, but are not limited to, halo, nitro, cyano, thiol, amino, hydroxy, carboxylic acid, sulfonic acid, aromatic group, and aromatic or non-aromatic heterocyclic group. Preferably the amino acid side chain is a C₁-C₁₀ straight or branched chain hydrocarbon, optionally substituted with a thiol, amino, hydroxy, carboxylic acid, aromatic group, or aromatic or non-aromatic heterocyclic group.

The amino acid ester can be an ester of a naturally occurring amino acid or a synthetically prepared amino acid. The amino acid can be a D-amino acid or an L-amino acid. Preferably, the amino acid ester is the ester of a naturally occurring amino acid. More, preferably, the amino acid ester is an ester of an amino acid selected from glycine, alanine, valine, leucine, isoleucine, phenylalanine, asparagine, glutamine, tryptophane, proline, serine, threonine, tyrosine, hydroxyproline, cysteine, methionine, aspartic acid, glutamic acid, lysine, arginine, and histidine.

The hydrocarbon group, R₁, can be any C₁ to C₂₂ hydrocarbon group. Representative C₁ to C₂₂ hydrocarbon groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, allyl, cyclopentyl, cyclohexyl, cis-9-hexadecenyl, cis-9-octadecenyl, cis, cis-9,12-octadecenyl, and cis, cis, cis-9,12,15-octadecatrienyl.

In one embodiment, R₁ is a straight chain hydrocarbon group.

In one embodiment, R₁ is a branched chain hydrocarbon group.

In one embodiment, R₁ is a saturated hydrocarbon group.

In one embodiment, R₁ is an unsaturated hydrocarbon group.

In one embodiment, R₁ is a straight chain, saturated hydrocarbon group.

In one embodiment, R₁ is a straight chain, unsaturated hydrocarbon group.

In one embodiment, R₁ is a C₁-C₁₆ hydrocarbon group.

In one embodiment, R₁ is a C₁-C₁₀ hydrocarbon group.

In one embodiment, R₁ is a C₁-C₅ hydrocarbon group.

In one embodiment, R₁ is a C₁-C₃ hydrocarbon group.

In one embodiment, R₁ is a C₆-C₂₂ hydrocarbon group.

In one embodiment, R₁ is a C₆-C₁₈ hydrocarbon group.

In one embodiment, R₁ is a C₈-C₁₈ hydrocarbon group.

In one embodiment, R₁ is a C₁₀-C₁₈ hydrocarbon group.

In one embodiment, R₁ is a C₁₆-C₁₈ hydrocarbon group.

In one embodiment, R₁ is a C₁₆-C₂₂ hydrocarbon group.

In one embodiment, R₁ is a C₁-C₁₆ straight chain hydrocarbon group.

In one embodiment, R₁ is a C₁-C₁₀ straight chain hydrocarbon group.

In one embodiment, R₁ is a C₁-C₅ straight chain hydrocarbon group.

In one embodiment, R₁ is a C₁-C₃ straight chain hydrocarbon group.

In one embodiment, R₁ is a C₆-C₂₂ straight chain hydrocarbon group.

In one embodiment, R₁ is a C₆-C₁₈ straight chain hydrocarbon group.

In one embodiment, R₁ is a C₈-C₁₈ straight chain hydrocarbon group.

In one embodiment, R₁ is a C₁₀-C₁₈ straight chain hydrocarbon group.

In one embodiment, R₁ is a C₁₆-C₁₈ straight chain hydrocarbon group.

In one embodiment, R₁ is a C₁₆-C₂₂ straight chain hydrocarbon group.

In one embodiment, R₁ is a C₁-C₁₆ branched chain hydrocarbon group.

In one embodiment, R₁ is a C₁-C₁₀ branched chain hydrocarbon group.

In one embodiment, R₁ is a C₁-C₅ branched chain hydrocarbon group.

In one embodiment, R₁ is a C₁-C₃ branched chain hydrocarbon group.

In one embodiment, R₁ is a C₆-C₂₂ branched chain hydrocarbon group.

In one embodiment, R₁ is a C₆-C₁₈ branched chain hydrocarbon group.

In one embodiment, R₁ is a C₈-C₁₈ branched chain hydrocarbon group.

In one embodiment, R₁ is a C₁₀-C₁₈ branched chain hydrocarbon group.

In one embodiment, R₁ is a C₁₆-C₁₈ branched chain hydrocarbon group.

In one embodiment, R₁ is a C₁₆-C₂₂ branched chain hydrocarbon group.

In one embodiment, R₁ is a C₁-C₁₆ straight chain unsaturated hydrocarbon group.

In one embodiment, R₁ is a C₁-C₁₀ straight chain unsaturated hydrocarbon group.

In one embodiment, R₁ is a C₁-C₅ straight chain unsaturated hydrocarbon group.

In one embodiment, R₁ is a C₁-C₃ straight chain unsaturated hydrocarbon group.

In one embodiment, R₁ is a C₆-C₂₂ straight chain unsaturated hydrocarbon group.

In one embodiment, R₁ is a C₆-C₁₈ straight chain unsaturated hydrocarbon group.

In one embodiment, R₁ is a C₈-C₁₈ straight chain unsaturated hydrocarbon group.

In one embodiment, R₁ is a C₁₀-C₁₈ straight chain unsaturated hydrocarbon group.

In one embodiment, R₁ is a C₁₆-C₁₈ straight chain unsaturated hydrocarbon group.

In one embodiment, R₁ is a C₁₆-C₂₂ straight chain unsaturated hydrocarbon group.

The amino acid esters can be obtained by esterifying an amino acid with an alcohol of formula R₁—OH using methods well known to those skilled in the art such as those described in J. March, Advanced Organic Chemistry, Reaction Mechanisms and Structure, 4^{th} ed. John Wiley & Sons, NY, 1992, pp. 393-400. The amino acids and alcohols of formula R₁—OH are commercially available or can be prepared by methods well known to those skilled in the art. When esterifying the amino acid with the alcohol of formula R₁—OH, it may be necessary to protect some other functional group of the amino acid or the alcohol with a protecting group that is subsequently removed after the esterification reaction. One of ordinary skill in the art would readily know what functional groups would need to be protected before esterifying the amino acid with the alcohol of formula R₁—OH. Suitable protecting groups are known to those skilled in the art such as those described in T. W. Greene, et al. Protective Groups in Organic Synthesis, 3^{rd} ed. (1999).

### 7.3.2 The amino acid amide

The amino acid amide can be any amide of any amino acid, i.e., an amino acid wherein the carboxylic acid group of the amino acid is reacted with an amine of formula HN(R₃)(R₄), wherein R₃ and R₄ are defined below, to provide an amide. Accordingly, the amino acid amides have the general formula (II):

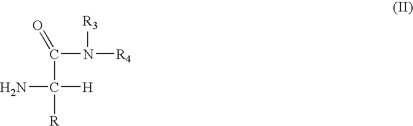

wherein

R is the amino acid side chain;

R₃ is hydrogen or a C₁ to C₂₂ hydrocarbon group; and

R₄ is hydrogen or a C₁ to C₂₂ hydrocarbon group.

As one of ordinary skill in the art would readily know, a wide variety of groups are possible for the amino acid side, R. For example, the amino acid side can be a hydrocarbon group that can be optionally substituted. Suitable substituents include, but are not limited to, halo, nitro, cyano, thiol, amino, hydroxy, carboxylic acid, sulfonic acid, aromatic group, and aromatic or non-aromatic heterocyclic group. Preferably the amino acid side chain is a C₁-C₁₀ straight or branched chain hydrocarbon, optionally substituted with a thiol, amino, hydroxy, carboxylic acid, aromatic group, or aromatic or non-aromatic heterocyclic group; an aromatic group, or an aromatic or non-aromatic heterocyclic group.

The amino acid amide can be an amide of a naturally occurring amino acid or a synthetically prepared amino acid. The amino acid can be a D-amino acid or an L-amino acid. Preferably, the amino acid amide is the amide of a naturally occurring amino acid. More, preferably, the amino acid amide is an amide of an amino acid selected from glycine, alanine, valine, leucine, isoleucine, phenylalanine, asparagine, glutamine, tryptophane, proline, serine, threonine, tyrosine, hydroxyproline, cysteine, methionine, aspartic acid, glutamic acid, lysine, arginine, and histidine.

The R₃ group can be hydrogen or any C₁ to C₂₂ hydrocarbon group. The R₄ group can be hydrogen or any C₁ to C₂₂ hydrocarbon group. Representative C₁ to C₂₂ hydrocarbon groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, allyl, cyclopentyl, cyclohexyl, cis-9-hexadecenyl, cis-9-octadecenyl, cis, cis-9,12-octadecenyl, and cis, cis, cis-9,12,15-octadecatrienyl.

In one embodiment, each of R₃ and R₄ is a hydrogen.

In one embodiment, R₄ is hydrogen and R₃ is a straight chain hydrocarbon group.

In one embodiment, R₄ is hydrogen and R₃ is a branched chain hydrocarbon group.

In one embodiment, R₄ is hydrogen and R₃ is a saturated hydrocarbon group.

In one embodiment, R₄ is hydrogen and R₃ is an unsaturated hydrocarbon group.

In one embodiment, R₄ is hydrogen and R₃ is a straight chain, saturated hydrocarbon group.

In one embodiment, R₄ is hydrogen and R₃ is a straight chain, unsaturated hydrocarbon group.

In one embodiment, R₄ is hydrogen and R₃ is a C₁-C₁₆ hydrocarbon group.

In one embodiment, R₄ is hydrogen and R₃ is a C₁-C₁₀ hydrocarbon group.

In one embodiment, R₄ is hydrogen and R₃ is a C₁-C₅ hydrocarbon group.

In one embodiment, R₄ is hydrogen and R₃ is a C₁-C₃ hydrocarbon group.

In one embodiment, R₄ is hydrogen and R₃ is a C₆-C₂₂ hydrocarbon group.

In one embodiment, R₄ is hydrogen and R₃ is a C₆-C₁₈ hydrocarbon group.

In one embodiment, R₄ is hydrogen and R₃ is a C₈-C₁₈ hydrocarbon group.

In one embodiment, R₄ is hydrogen and R₃ is a C₁₀-C₁₈ hydrocarbon group.

In one embodiment, R₄ is hydrogen and R₃ is a C₁₆-C₁₈ hydrocarbon group.

In one embodiment, R₄ is hydrogen and R₃ is a C₁₆-C₂₂ hydrocarbon group.

In one embodiment, R₄ is hydrogen and R₃ is a C₁-C₁₆ straight chain hydrocarbon group.

In one embodiment, R₄ is hydrogen and R₃ is a C₁-C₁₀ straight chain hydrocarbon group.

In one embodiment, R₄ is hydrogen and R₃ is a C₁-C₅ straight chain hydrocarbon group.

In one embodiment, R₄ is hydrogen and R₃ is a C₁-C₃ straight chain hydrocarbon group.

In one embodiment, R₄ is hydrogen and R₃ is a C₆-C₂₂ straight chain hydrocarbon group.

In one embodiment, R₄ is hydrogen and R₃ is a C₆-C₁₈ straight chain hydrocarbon group.

In one embodiment, R₄ is hydrogen and R₃ is a C₈-C₁₈ straight chain hydrocarbon group.

In one embodiment, R₄ is hydrogen and R₃ is a C₁₀-C₁₈ straight chain hydrocarbon group.

In one embodiment, R₄ is hydrogen and R₃ is a C₁₆-C₁₈ straight chain hydrocarbon group.

In one embodiment, R₄ is hydrogen and R₃ is a C₁₆-C₂₂ straight chain hydrocarbon group.

In one embodiment, R₄ is hydrogen and R₃ is a C₁-C₁₆ branched chain hydrocarbon group.

In one embodiment, R₄ is hydrogen and R₃ is a C₁-C₁₀ branched chain hydrocarbon group.

In one embodiment, R₄ is hydrogen and R₃ is a C₁-C₅ branched chain hydrocarbon group.

In one embodiment, R₄ is hydrogen and R₃ is a C₁-C₃ branched chain hydrocarbon group.

In one embodiment, R₄ is hydrogen and R₃ is a C₆-C₂₂ branched chain hydrocarbon group.

In one embodiment, R₄ is hydrogen and R₃ is a C₆-C₁₈ branched chain hydrocarbon group.

In one embodiment, R₄ is hydrogen and R₃ is a C₈-C₁₈ branched chain hydrocarbon group.

In one embodiment, R₄ is hydrogen and R₃ is a C₁₀-C₁₈ branched chain hydrocarbon group.

In one embodiment, R₄ is hydrogen and R₃ is a C₁₆-C₁₈ branched chain hydrocarbon group.

In one embodiment, R₄ is hydrogen and R₃ is a C₁₆-C₂₂ branched chain hydrocarbon group.

In one embodiment, R₄ is hydrogen and R₃ is a C₁-C₁₆ straight chain saturated hydrocarbon group.

In one embodiment, R₄ is hydrogen and R₃ is a C₁-C₁₀ straight chain saturated hydrocarbon group.

In one embodiment, R₄ is hydrogen and R₃ is a C₁-C₅ straight chain saturated hydrocarbon group.

In one embodiment, R₄ is hydrogen and R₃ is a C₁-C₃ straight chain saturated hydrocarbon group.

In one embodiment, R₄ is hydrogen and R₃ is a C₆-C₂₂ straight chain saturated hydrocarbon group.

In one embodiment, R₄ is hydrogen and R₃ is a C₆-C₁₈ straight chain saturated hydrocarbon group.

In one embodiment, R₄ is hydrogen and R₃ is a C₈-C₁₈ straight chain saturated hydrocarbon group.

In one embodiment, R₄ is hydrogen and R₃ is a C₁₀-C₁₈ straight chain saturated hydrocarbon group.

In one embodiment, R₄ is hydrogen and R₃ is a C₁₆-C₁₈ straight chain saturated hydrocarbon group.

In one embodiment, R₄ is hydrogen and R₃ is a C₁₆-C₂₂ straight chain saturated hydrocarbon group.

In one embodiment, each of R₃ and R₄ are a straight or branched chain, saturated or unsaturated hydrocarbon group, wherein R₃ and R₄ may be the same or different.

In one embodiment, each of R₃ and R₄ are a C₁-C₁₆ hydrocarbon group, wherein R₃ and R₄ may be the same or different.

In one embodiment, each of R₃ and R₄ are a C₁-C₁₀ hydrocarbon group, wherein R₃ and R₄ may be the same or different.

In one embodiment, each of R₃ and R₄ are a C₁-C₅ hydrocarbon group, wherein R₃ and

R₄ may be the same or different.

In one embodiment, each of R₃ and R₄ are a C₁-C₃ hydrocarbon group, wherein R₃ and

R₄ may be the same or different.

In one embodiment, each of R₃ and R₄ are a C₆-C₂₂ hydrocarbon group, wherein R₃ and R₄ may be the same or different.

In one embodiment, each of R₃ and R₄ are a C₆-C₁₈ hydrocarbon group, wherein R₃ and R₄ may be the same or different.

In one embodiment, each of R₃ and R₄ are a C₈-C₁₈ hydrocarbon group, wherein R₃ and R₄ may be the same or different.

In one embodiment, each of R₃ and R₄ are a C₁₀-C₁₈ hydrocarbon group, wherein R₃ and R₄ may be the same or different.

In one embodiment, each of R₃ and R₄ are a C₁₆-C₁₈ hydrocarbon group, wherein R₃ and R₄ may be the same or different.

In one embodiment, each of R₃ and R₄ are a C₁₆-C₂₂ hydrocarbon group, wherein R₃ and R₄ may be the same or different.

In one embodiment, each of R₃ and R₄ are a C₁-C₁₆ straight chain hydrocarbon group, wherein R₃ and R₄ may be the same or different.

In one embodiment, each of R₃ and R₄ are a C₁-C₁₀ straight chain hydrocarbon group, wherein R₃ and R₄ may be the same or different.

In one embodiment, each of R₃ and R₄ are a C₁-C₅ straight chain hydrocarbon group, wherein R₃ and R₄ may be the same or different.

In one embodiment, each of R₃ and R₄ are a C₁-C₃ straight chain hydrocarbon group, wherein R₃ and R₄ may be the same or different.

In one embodiment, each of R₃ and R₄ are a C₆-C₂₂ straight chain hydrocarbon group, wherein R₃ and R₄ may be the same or different.

In one embodiment, each of R₃ and R₄ are a C₆-C₁₈ straight chain hydrocarbon group, wherein R₃ and R₄ may be the same or different.

In one embodiment, each of R₃ and R₄ are a C₈-C₁₈ straight chain hydrocarbon group, wherein R₃ and R₄ may be the same or different.

In one embodiment, each of R₃ and R₄ are a C₁₀-C₁₈ straight chain hydrocarbon group, wherein R₃ and R₄ may be the same or different.

In one embodiment, each of R₃ and R₄ are a C₁₆-C₁₈ straight chain hydrocarbon group, wherein R₃ and R₄ may be the same or different.

In one embodiment, each of R₃ and R₄ are a C₁₆-C₂₂ straight chain hydrocarbon group, wherein R₃ and R₄ may be the same or different.

In one embodiment, each of R₃ and R₄ are a C₁-C₁₆ branched chain hydrocarbon group, wherein R₃ and R₄ may be the same or different.

In one embodiment, each of R₃ and R₄ are a C₁-C₁₀ branched chain hydrocarbon group, wherein R₃ and R₄ may be the same or different.

In one embodiment, each of R₃ and R₄ are a C₁-C₅ branched chain hydrocarbon group, wherein R₃ and R₄ may be the same or different.

In one embodiment, each of R₃ and R₄ are a C₁-C₃ branched chain hydrocarbon group, wherein R₃ and R₄ may be the same or different.

In one embodiment, each of R₃ and R₄ are a C₆-C₂₂ branched chain hydrocarbon group, wherein R₃ and R₄ may be the same or different.

In one embodiment, each of R₃ and R₄ are a C₆-C₁₈ branched chain hydrocarbon group, wherein R₃ and R₄ may be the same or different.

In one embodiment, each of R₃ and R₄ are a C₈-C₁₈ branched chain hydrocarbon group, wherein R₃ and R₄ may be the same or different.

In one embodiment, each of R₃ and R₄ are a C₁₀-C₁₈ branched chain hydrocarbon group, wherein R₃ and R₄ may be the same or different.

In one embodiment, each of R₃ and R₄ are a C₁₆-C₁₈ branched chain hydrocarbon group, wherein R₃ and R₄ may be the same or different.

In one embodiment, each of R₃ and R₄ are a C₁₆-C₂₂ branched chain hydrocarbon group, wherein R₃ and R₄ may be the same or different.

In one embodiment, each of R₃ and R₄ are a C₁-C₁₆ straight chain saturated hydrocarbon group, wherein R₃ and R₄ may be the same or different.

In one embodiment, each of R₃ and R₄ are a C₁-C₁₀ straight chain saturated hydrocarbon group, wherein R₃ and R₄ may be the same or different.

In one embodiment, each of R₃ and R₄ are a C₁-C₅ straight chain saturated hydrocarbon group, wherein R₃ and R₄ may be the same or different.

In one embodiment, each of R₃ and R₄ are a C₁-C₃ straight chain saturated hydrocarbon group, wherein R₃ and R₄ may be the same or different.

In one embodiment, each of R₃ and R₄ are a C₆-C₂₂ straight chain saturated hydrocarbon group, wherein R₃ and R₄ may be the same or different.

In one embodiment, each of R₃ and R₄ are a C₆-C₁₈ straight chain saturated hydrocarbon group, wherein R₃ and R₄ may be the same or different.

In one embodiment, each of R₃ and R₄ are a C₈-C₁₈ straight chain saturated hydrocarbon group, wherein R₃ and R₄ may be the same or different.

In one embodiment, each of R₃ and R₄ are a C₁₀-C₁₈ straight chain saturated hydrocarbon group, wherein R₃ and R₄ may be the same or different.

In one embodiment, each of R₃ and R₄ are a C₁₆-C₁₈ straight chain saturated hydrocarbon group, wherein R₃ and R₄ may be the same or different.

In one embodiment, each of R₃ and R₄ are a C₁₆-C₂₂ straight chain saturated hydrocarbon group, wherein R₃ and R₄ may be the same or different.

In one embodiment, the combined number of carbon atoms in R₃ and R₄ is at least 6. In one embodiment, the combined number of carbon atoms in R₃ and R₄ is at least 8. In one embodiment, the combined number of carbon atoms in R₃ and R₄ is at least 10. In one embodiment, the combined number of carbon atoms in R₃ and R₄ is at least 12. In one embodiment, the combined number of carbon atoms in R₃ and R₄ is at least 18.

In one embodiment, the combined number of carbon atoms in R₃ and R₄ is less than 6. In one embodiment, the combined number of carbon atoms in R₃ and R₄ is less than 8. In one embodiment, the combined number of carbon atoms in R₃ and R₄ is less than 10. In one embodiment, the combined number of carbon atoms in R₃ and R₄ is less than 12. In one embodiment, the combined number of carbon atoms in R₃ and R₄ is less than 18.

In one embodiment, the combined number of carbon atoms in R₃ and R₄ ranges from about 1 to 16. In one embodiment, the combined number of carbon atoms in R₃ and R₄ ranges from about 1 to 10. In one embodiment, the combined number of carbon atoms in R₃ and R₄ ranges from about 1 to 5. In one embodiment, the combined number of carbon atoms in R₃ and R₄ ranges from about 1 to 3. In one embodiment, the combined number of carbon atoms in R₃ and R₄ ranges from about 16 to 22. In one embodiment, the combined number of carbon atoms in R₃ and R₄ ranges from about 16 to 18. In one embodiment, the combined number of carbon atoms in R₃ and R₄ ranges from about 8 to 18. In one embodiment, the combined number of carbon atoms in R₃ and R₄ ranges from about 10 to 18. In one embodiment, the combined number of carbon atoms in R₃ and R₄ ranges from about 12 to 18. In one embodiment, the combined number of carbon atoms in R₃ and R₄ ranges from about 6 to 30. In one embodiment, the combined number of carbon atoms in R₃ and R₄ ranges from about 22 to 30.

The amino acid amides can be obtained by converting the carboxylic acid group of the amino acid to an amide group using methods well known to those skilled in the art such as those described in J. March, Advanced Organic Chemistry, Reaction Mechanisms and Structure, 4^{th} ed. John Wiley & Sons, NY, 1992, pp. 417-427. Typically, the amino acid is converted to an amino acid derivative such as an amino acid ester or an acid chloride of the amino acid and the amino acid derivative is then reacted with an amine of formula NHR₃R₄ to provide the amino acid amide. The amino acids and amines of formula NHR₃R₄ are commercially available or can be prepared by methods well known to those skilled in the art. When forming the derivative of the amino acid or reacting the amino acid derivative with an amine of formula NHR₃R₄, it may be necessary to protect some other functional group of the amino acid derivative or the amine with a protecting group that is subsequently removed after the amidation reaction. One of ordinary skill in the art would readily know what functional groups would need to be protected before reacting the derivative of the amino acid with the amine of formula NHR₃R₄. Suitable protecting groups are known to those skilled in the art such as those described in T. W. Greene, et al. Protective Groups in Organic Synthesis, 3^{rd} ed. (1999).

### 7.3.3 The Amino Acid-Vitamin Ester

The amino acid-vitamin esters are esters formed between an amino acid and a vitamin that contains a hydroxyl group, i.e., an amino acid wherein the carboxylic acid group of the amino acid is esterified with the hydroxyl group (i.e., —OH group) of the vitamin. Accordingly, the amino acid-vitamin esters have the general formula:

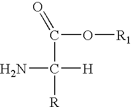

wherein

R is the amino acid side chain; and

O—R₁ is the residue of a vitamin.

As one of ordinary skill in the art would readily know, a wide variety of groups are possible for the amino acid side, R. For example, the amino acid side can be a hydrocarbon group that can be optionally substituted. Suitable substituents include, but are not limited to, halo, nitro, cyano, thiol, amino, hydroxy, carboxylic acid, sulfonic acid, aromatic group, and aromatic or non-aromatic heterocyclic group. Preferably the amino acid side chain is a C₁-C₁₀ straight or branched chain hydrocarbon, optionally substituted with a thiol, amino, hydroxy, carboxylic acid, aromatic group, or non-aromatic heterocyclic group; an aromatic group, or an aromatic or non-aromatic heterocyclic group.

The amino acid of the amino acid-vitamin ester can be a naturally occurring amino acid or a synthetically prepared amino acid. The amino acid can be a D-amino acid or an amino acid. Preferably, the amino acid-vitamin ester is the ester of a naturally occurring amino acid. More preferably, the amino acid-vitamin ester is an ester of an amino acid selected from glycine, alanine, valine, leucine, isoleucine, phenylalanine, asparagine, glutamine, tryptophane, proline, serine, threonine, tyrosine, hydroxyproline, cysteine, methionine, aspartic acid, glutamic acid, lysine, arginine, and histidine.

The vitamin can be any vitamin that includes a hydroxyl group. Illustrative vitamins include, but are not limited to, vitamin A (retinol), vitamin B₁ (thiamin), vitamin B₂ (riboflavin), vitamin B₅ (pantothenic acid), vitamin B₆, vitamin B₁₂ (cyanocobalamin), vitamin C, vitamin D, and vitamin E.

In one embodiment, the vitamin is vitamin A.

In one embodiment, the vitamin is vitamin B₁.

In one embodiment, the vitamin is vitamin B₂.

In one embodiment, the vitamin is vitamin B₅.

In one embodiment, the vitamin is vitamin B₆.

In one embodiment, the vitamin is vitamin B₁₂.

In one embodiment, the vitamin is vitamin C.

In one embodiment, the vitamin is vitamin D.

In one embodiment, the vitamin is vitamin E.

The amino acid-vitamin esters can be obtained by esterifying an amino acid with a vitamin of formula R₁—OH using methods well known to those skilled in the art such as those described in J. March, Advanced Organic Chemistry, Reaction Mechanisms and Structure, 4^{th} ed. John Wiley & Sons, NY, 1992, pp. 393-400. The amino acids and vitamins are commercially available or can be prepared by methods well known to those skilled in the art. When esterifying the amino acid with the vitamin, it may be necessary to protect some other functional group of the amino acid or the vitamin with a protecting group that is subsequently removed after the esterification reaction. One of ordinary skill in the art would readily know what functional groups would need to be protected before esterifying the amino acid with the vitamin. Suitable protecting groups are known to those skilled in the art such as those described in T. W. Greene, et al. Protective Groups in Organic Synthesis, 3^{rd} ed. (1999).

### 7.4 Examples of Pharmaceutical Compositions of the Invention

### 7.4.1 Pharmaceutical Compositions Comprising Nano-Particles or Micro-Particles Comprising (i) a Pharmaceutically Acceptable Organic Base and (ii) a Protonated Oligonucleotide

In one embodiment, the pharmaceutical composition comprises nano-particles or micro-particles comprising (i) a protonated oligonucleotide and an (ii) a pharmaceutically acceptable organic base. In one embodiment, the particles are nano-particles. In one embodiment, the particles are micro-particles.

Without wishing to be bound by theory, it is believed that the acidic phosphate groups of the a protonated oligonucleotide protonates the amine group of the pharmaceutically acceptable organic base to form a salt between one or more pharmaceutically acceptable organic base molecules and the oligonucleotide as illustrated schematically below for a pharmaceutically acceptable organic base of formula Base-NH₂ and a protonated aptamer.

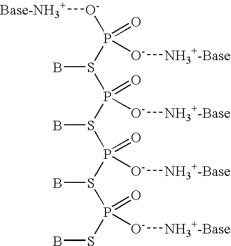

wherein B is a nucleotide, S is a sugar, and Base-NH₃⁺ is a protonated pharmaceutically acceptable organic base. It is not necessary, however, that every phosphate group be ionically bound to a pharmaceutically acceptable organic base molecule.

Any pharmaceutically acceptable organic base described above can be used in the pharmaceutical compositions.

Any oligonucleotide described above can be used in the pharmaceutical compositions.

The molar ratio of acidic groups on the oligonucleotide to basic groups on the a pharmaceutically acceptable organic base typically ranges from about 2:1 to 1:2. In one embodiment, the molar ratio of acidic groups on the oligonucleotide to basic groups on the pharmaceutically acceptable organic base ranges about 1.5:1 to 1:1.5. In one embodiment, the molar ratio of acidic groups on the oligonucleotide to basic groups on the pharmaceutically acceptable organic base ranges about 1.25:1 to 1:1.25. In one embodiment, the molar ratio of acidic groups on the oligonucleotide to basic groups on the pharmaceutically acceptable organic base ranges about 1.1:1. to 1:1.1. In one embodiment, the molar ratio of acidic groups on the oligonucleotide to basic groups on the pharmaceutically acceptable organic base is about 1:1. A wider range for the molar ratio of acidic groups on the oligonucleotide to basic groups on the pharmaceutically acceptable organic base, however, is also possible. For example, the molar ratio of acidic groups on the oligonucleotide to basic groups on the pharmaceutically acceptable organic base can range from about 15:1 to 1:15.

### 7.4.1 (i) Pharmaceutical Compositions Comprising Nano-Particles Comprising (i) an Amino Acid Ester or Amino Acid Amide and (ii) a Protonated Oligonucleotide

Without wishing to be bound by theory, it is believed that the acidic phosphate groups of the protonated oligonucleotide protonate the amine group of the amino acid ester or amide to form a salt between one or more amino acid ester or amide molecules and the oligonucleotide as illustrated schematically below for an amino acid ester and an aptamer:

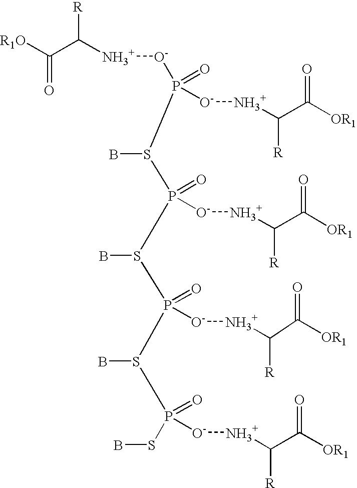

wherein B, S, R, and R₁ have the meaning described above. It is not necessary, however, that every phosphate group be ionically bound to an amino acid ester or amino acid amide.

In one embodiment, the particles are nano-particles. In one embodiment, the particles are micro-particles.

Any amino acid or amino acid ester described above can be used in the pharmaceutical compositions.

In one embodiment, the amino acid ester is an amino acid vitamin ester, i.e., —OR₁ is the residue of a vitamin.

Any oligonucleotide described above can be used in the pharmaceutical compositions.

The molar ratio of acidic groups on the oligonucleotide to basic groups on the amino acid ester or amino acid amide typically ranges from about 2:1 to 1:2. In one embodiment, the molar ratio of acidic groups on the oligonucleotide to basic groups on the amino acid ester or amino acid amide ranges from about 1.5:1 to 1:1.5. In one embodiment, the molar ratio of acidic groups on the oligonucleotide to basic groups on the amino acid ester or amino acid amide ranges from about 1.25:1 to 1:1.25. In one embodiment, the molar ratio of acidic groups on the oligonucleotide to basic groups on the amino acid ester or amino acid amide ranges from about 1.1:1. to 1:1.1. In one embodiment, the molar ratio of acidic groups on the oligonucleotide to basic groups on the amino acid ester or amino acid amide is about 1:1. A wider range for the molar ratio of acidic groups on the oligonucleotide to basic groups on the amino acid ester or amino acid, however, is also possible. For example, the molar ratio of acidic groups on the oligonucleotide to basic groups on the amino acid ester or amino acid can range from about 15:1 to 1:15.

### 7.4.1 (i)(a) Pharmaceutical Compositions Comprising Nano-Particles or Micro-Particles Comprising (i) an Amino Acid Ester and (ii) a Protonated Oligonucleotide Wherein the Amino Acid Ester is an Amino Acid-Vitamin Ester

In one embodiment, the amino acid ester is an amino acid-vitamin ester, i.e., —OR₁ is the residue of a vitamin.

Any amino acid-vitamin ester described above can described above can be used in the pharmaceutical compositions.

Amino acid-vitamin esters are advantageous. The vitamin part of the amino acid-vitamin ester nano-particle or micro-particle can be used as a means for the nano-particle or micro-particle to interact with proteins, such as transfer proteins (for example, tocopherol transfer protein), found in the serum. This interaction between the vitamin and a protein can advantageously extends the t_{1/2} of the composition when it is administered to an animal.

### 7.4.1 (i)(b) Pharmaceutical Compositions Comprising Nano-Particles or Micro-Particles Comprising (i) an Amino Acid Ester or Amino Acid Amide and (ii) a Protonated Oligonucleotide Wherein the Amino Acid Ester or Amide is an Amino Acid Ester or Amide of Lysine

In one embodiment, the pharmaceutical composition comprises an ester or amide of lysine.

In one embodiment, there is less than a molar equivalent of lysine molecules relative to acidic phosphate groups on the oligonucleotide, i.e., there is an excess of acidic phosphate groups on the oligonucleotide relative to amino acid ester or amide molecules.

Without wishing to be bound by theory it is believed that the amino acid ester or amide of lysine cross-links two protonated oligonucleotide molecules as depicted below:

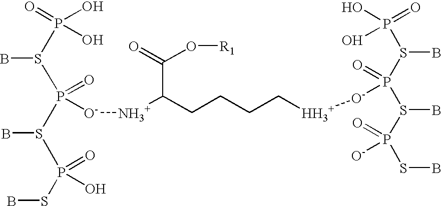

wherein B, S, and R₁ is a C₁-C₂₁ hydrocarbon group.

### Pharmaceutical Compositions Comprising an Ester or Amide of Lysine, a Protonated Aptamer, and a Carboxylic Acid

In one embodiment, the amino acid ester or amide is an ester or amide of lysine and the pharmaceutical composition further comprises a carboxylic acid. Without wishing to be bound by theory, it is believed that the carboxylic acid protonates the s-amine group of lysine to provide a structure as depicted below:

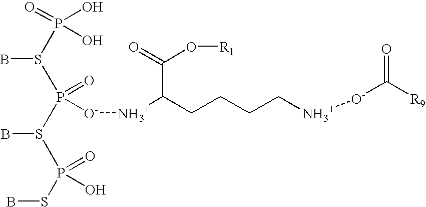

wherein B, S, and R₁ and R₉ are each independently a C₁-C₂₁ hydrocarbon group.

The combined molar ratio of acidic groups on the oligonucleotide and acid groups on the carboxylic acid to basic groups on the amino acid ester or amino acid amide typically ranges from about 2:1 to 1:2. In one embodiment, the combined molar ratio of acidic groups on the oligonucleotide and acid groups on the carboxylic acid to basic groups on the amino acid ester or amino acid amide ranges from about 1.5:1 to 1:1.5. In one embodiment, the combined molar ratio of acidic groups on the oligonucleotide and acid groups on the carboxylic acid to basic groups on the amino acid ester or amino acid amide ranges from about 1.25:1 to 1:1.25. In one embodiment, the combined molar ratio of acidic groups on the oligonucleotide and acid groups on the carboxylic acid to basic groups on the amino acid ester or amino acid amide ranges from about 1.1:1. to 1:1.1. In one embodiment, the combined molar ratio of acidic groups on the oligonucleotide and acid groups on the carboxylic acid to basic groups on the amino acid ester or amino acid amide is about 1:1. A wider range for the molar ratio of acidic groups on the oligonucleotide and acid groups on the carboxylic acid to basic groups on the amino acid ester or amino acid amide, however, is also possible. For example, the molar ratio of acidic groups on the oligonucleotide and acid groups on the carboxylic acid to basic groups on the amino acid ester or amino acid amide can range from about 20:1 to 1:20. In one embodiment, the molar ratio of acidic groups on the oligonucleotide to acid groups on the carboxylic acid ranges from about 15:1 to 1:15. In one embodiment, the molar ratio of acidic groups on the oligonucleotide to acid groups on the carboxylic acid ranges from about 10:1 to 1:10. In one embodiment, the molar ratio of acidic groups on the oligonucleotide to acid groups on the carboxylic acid ranges from about 5:1 to 1:5.

### The Carboxylic Acid

The carboxylic acid can be any pharmaceutically acceptable carboxylic acid. Typically, the carboxylic acid is a C₁-C₂₂ carboxylic acid. Suitable carboxylic acids include, but are not limited to, acetic acid, propanoic acid, butanoic acid, pentanoic acid, decanoic acid, hexanoic acid, benzoic acid, caproic acid, lauric acid, myristic acid, palmitic acid, stearic acid, palmic acid, oleic acid, linoleic acid, and linolenic acid.

In one embodiment, the carboxylic acid is a C₁-C₁₆ carboxylic acid.

In one embodiment, the carboxylic acid is a C₁-C₁₀ carboxylic acid.

In one embodiment, the carboxylic acid is a C₁-C₅ carboxylic acid.

In one embodiment, the carboxylic acid is a C₁-C₃ carboxylic acid.

In one embodiment, the carboxylic acid is a C₆-C₂₂ carboxylic acid.

In one embodiment, the carboxylic acid is a C₆-C₁₈ carboxylic acid.

In one embodiment, the carboxylic acid is a C₈-C₁₈ carboxylic acid.

In one embodiment, the carboxylic acid is a C₁₀-C₁₈ carboxylic acid.

In one embodiment, the carboxylic acid is a C₆-C₁₈ carboxylic acid.

In one embodiment, the carboxylic acid is a C₁₆-C₂₂ carboxylic acid.

In one embodiment, the carboxylic acid is a saturated or unsaturated fatty acid.

In one embodiment, the carboxylic acid is a saturated fatty acid.

In one embodiment, the carboxylic acid is an unsaturated fatty acid.

In one embodiment, the carboxylic acid is a dicarboxylic acid. Suitable dicarboxylic acids include, but are not limited to, oxalic acid, malonic aid, succinic acid, glutamic acid, adipic acid, and pimelic acid.

In one embodiment, the carboxylic acid is a polycarboxylic acid.

The carboxylic acids are commercially available or can be prepared by methods well known to those skilled in the art.

In one embodiment, the carboxylic acid is an N-acyl amino acid. The N-acyl amino acids have the following general formula (III):

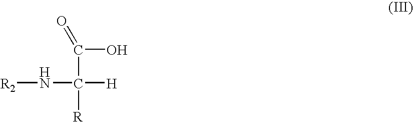

wherein:

R is the amino acid side chain and is defined above; and

R₂ is an acyl group of formula —C(O)—R₅, wherein R₅ is a substituted C₁ to C₂₁ hydrocarbon group, i.e., the acyl group, R₂, is a C₁ to C₂₂ acyl group. Representative acyl groups of formula —C(O)—R₅ include, but are not limited to, acetyl, propionyl, butanoyl, hexanoyl, caproyl, heptoyl, octoyl, nonoyl, decoyl, undecoyl, dodecoyl, tridecoyl, tetradecoyl, pentadecoyl, hexadecoyl, heptadecoyl, octadecoyl, laurolyl, myristoyl, palmitoyl, stearoyl, palmioleoyl, oleoyl, linoleoyl, linolenoyl, and benzoyl.

In one embodiment, R₅ is a C₁-C₁₅ hydrocarbon group, i.e., the acyl group of formula —C(O)—R₅ is a C₂-C₁₆ acyl group.

In one embodiment, R₅ is a C₁-C₉ hydrocarbon group, i.e., the acyl group of formula —C(O)—R₅ is a C₂-C₁₀ acyl group.

In one embodiment, R₅ is a C₁-C₅ hydrocarbon group, i.e., the acyl group of formula —C(O)—R₅ is a C₂-C₆ acyl group.

In one embodiment, R₅ is a C₁-C₃ hydrocarbon group, i.e., the acyl group of formula —C(O)—R₅ is a C₂-C₄ acyl group.

In one embodiment, R₅ is a C₅-C₂₁ hydrocarbon group, i.e., the acyl group of formula —C(O)—R₅ is a C₆-C₂₂ acyl group.

In one embodiment, R₅ is a C₅-C₁₇ hydrocarbon group, i.e., the acyl group of formula —C(O)—R₅ is a C₆-C₁₈ acyl group.

In one embodiment, R₅ is a C₇-C₁₇ hydrocarbon group, i.e., the acyl group of formula —C(O)—R₅ is a C₈-C₁₈ acyl group.

In one embodiment, R₅ is a C₉-C₁₇ hydrocarbon group, i.e., the acyl group of formula —C(O)—R₅ is a C₁₀-C₁₈ acyl group.

In one embodiment, R₅ is a C₁₅-C₂₁ hydrocarbon group, i.e., the acyl group of formula —C(O)—R₅ is a C₁₆-C₂₂ acyl group.

In one embodiment, the acyl group of formula —C(O)—R₅ is obtained from a saturated or unsaturated fatty acid.

In one embodiment, the acyl group of formula —C(O)—R₅ is a caproyl, laurolyl, myristoyl, palmitoyl, stearoyl, palmioleoyl, oleoyl, linoleoyl, or linolenoyl group.

The N-acylated amino acids can be obtained by methods well known to those skilled in the art. For example, the N-acylated amino acids can be obtained by reacting an amino acid with an acid halide of formula T-C(O)—R₅, wherein T is a halide, preferably chloride, and R₁ is as defined above, using methods well known to those skilled in the art. When N-acylating the amino acid with the acid halide of formula T-C(O)—R₅, it may be necessary to protect some other functional group of the amino acid or the acid halide with a protecting group that is subsequently removed after the acylation reaction. One of ordinary skill in the art would readily know what functional groups would need to be protected before acylating the amino acid with the acid halide of formula T-C(O)—R₅. Suitable protecting groups are known to those skilled in the art such as those described in T. W. Greene, et al. Protective Groups in Organic Synthesis, 3rd ed. (1999).

Acid halides can be obtained using methods well known to those skilled in the art such as those described in J. March, Advanced Organic Chemistry, Reaction Mechanisms and Structure, 4th ed. John Wiley & Sons, NY, 1992, pp. 437-8. For example, acid halides can be prepared by reacting a carboxylic acid with thionyl chloride, bromide, or iodide. Acid chlorides and bromides can also be prepared by reacting a carboxylic acid with phosphorous trichloride or phosphorous tribromide, respectively. Acid chlorides can also be prepared by reacting a carboxylic acid with Ph₃P in carbon tetrachloride. Acid fluorides can be prepared by reacting a carboxylic acid with cyanuric fluoride.

### Pharmaceutical Compositions Comprising an Ester or Amide of Lysine, a Protonated Oligonucleotide, and a Phospholipid, Phosphatidyl Choline, or a Sphingomyelin

In another embodiment, the amino acid ester or amide is an ester or amide of lysine and the pharmaceutical composition further comprises a phospholipid, phosphatidyl choline, or a sphingomyelin. Without wishing to be bound by theory, it is believed that protonated phosphate groups on the phospholipid, phosphatidyl choline, or sphingomyelin protonates the 8-amine group of lysine to provide a structure as depicted below for a phospholipid:

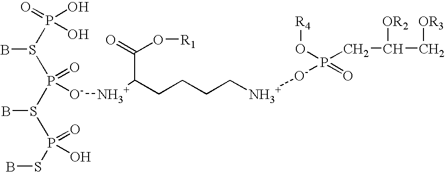

wherein B, S, R₁, R₂, R₃, and R₄ are defined above.

The combined molar ratio of acidic groups on the oligonucleotide and acidic groups on the phospholipid, phosphatidyl choline, or sphingomyelin to basic groups on the amino acid ester or amino acid amide typically ranges from about 2:1 to 1:2. In one embodiment, the combined molar ratio of acidic groups on the oligonucleotide and acidic groups on the phospholipid, phosphatidyl choline, or sphingomyelin to basic groups on the amino acid ester or amino acid amide ranges from about 1.5:1 to 1:1.5. In one embodiment, the combined molar ratio of acidic groups on the oligonucleotide and acidic groups on the phospholipid, phosphatidyl choline, or sphingomyelin to basic groups on the amino acid ester or amino acid amide ranges from about 1.25:1 to 1:1.25. In one embodiment, the combined molar ratio of acidic groups on the oligonucleotide and acidic groups on the phospholipid, phosphatidyl choline, or sphingomyelin to basic groups on the amino acid ester or amino acid amide ranges from about 1.1:1. to 1:1.1. In one embodiment, the combined molar ratio of acidic groups on the oligonucleotide and acidic groups on the phospholipid, phosphatidyl choline, or sphingomyelin to basic groups on the amino acid ester or amino acid amide is about 1:1. A wider range for the molar ratio of acidic groups on the oligonucleotide and acidic groups on the phospholipid, phosphatidyl choline, or sphingomyelin to basic groups on the amino acid ester or amino acid amide, however, is also possible. For example, the molar ratio of acidic groups on the oligonucleotide and acidic groups on the phospholipid, phosphatidyl choline, or sphingomyelin to basic groups on the amino acid ester or amino acid amide can range from about 20:1 to 1:20. In one embodiment, the molar ratio of acidic groups on the oligonucleotide to acidic groups on the phospholipid, phosphatidyl choline, or sphingomyelin ranges from about 15:1 to 1:15. In one embodiment, the molar ratio of acidic groups on the oligonucleotide to acidic groups on the phospholipid, phosphatidyl choline, or sphingomyelin ranges from about 10:1 to 1:10. In one embodiment, the molar ratio of acidic groups on the oligonucleotide to acidic groups on the phospholipid, phosphatidyl choline, or sphingomyelin ranges from, about 5:1 to 1:5.

### The phospholipid

Any pharmaceutically acceptable phospholipid can be used in the pharmaceutical compositions of the invention.

Representative, pharmaceutically acceptable phospholipids include, but are not limited to:

phosphatidic acids of general formula:

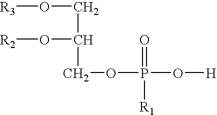

wherein R₁, R₂, and R₃ are defined above. Suitable phosphatidic acids suitable for use in the compositions and methods of the invention include, but are not limited to, the 1-acyl-2-acyl-sn-glycero-3-phosphates and the 1,2-diacyl-sn-glycero-3-phosphates commercially available from Avanti Polar Lipids Inc. of Alabaster, Ala.

phosphatidylethanolamines of general formula

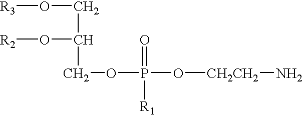

wherein R₁, R₂, and R₃ are defined above. Suitable phosphatidylethanolamines suitable for use in the compositions and methods of the invention include, but are not limited to, the 1-acyl-2-acyl-sn-glycero-3-phosphoethanolamines and the 1,2-diacyl-sn-glycero-3-phosphoethanolamines commercially available from Avanti Polar Lipids Inc. of Alabaster, Ala.

phosphatidylcholines of general formula

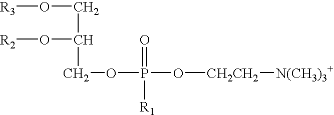

wherein R₁, R₂, and R₃ are defined above. Suitable phosphatidyleholines suitable for use in the compositions and methods of the invention include, but are not limited to, the 1-acyl-2-acyl-sn-glycero-3-phosphocholines, the 1,2-diacyl-sn-glycero-3-phosphoethanolamines (saturated series), and the 1,2-diacyl-sn-glycero-3-phosphoethanolamines (unsaturated series), commercially available from Avanti Polar Lipids Inc. of Alabaster, Ala. and Phospholipon®-50PG, Phospholipon®-53MCT, Phospholipon -75SA, Phospholipon®-80, Phospholipon®-90NG, Phospholipon®-90H, and Phospholipon®-100H, commercially available from Phospholipid GmbH of Cologne, Germany. In one embodiment, the phospholipid is Phospholipon®-90H.

phosphatidylserines of general formula

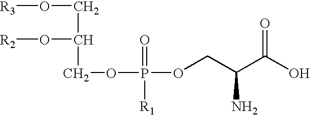

wherein R₁, R₂, and R₃ are defined above. Suitable phosphatidylserines suitable for use in the compositions and methods of the invention include, but are not limited to, the 1-acyl-2-acyl-sn-glycero-3-[phospho-L-serine]s and the 1,2-diacyl-sn-glycero-3-[phospho-L-serine]s commercially available from Avanti Polar Lipids Inc. of Alabaster, Ala.

plasmalogens of general formula

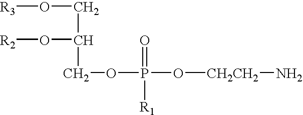

wherein R₁ and R₂ are defined above and R₃ is —C═C—R₉, wherein R₉ is defined above. Suitable plasmalogens suitable for use in the compositions and methods of the invention include, but are not limited to, C16(Plasm)-12:0 NBD PC, C16(Plasm)-18:1 PC, C16(Plasm)-20:4 PC, C16(Plasm)-22:6 PC, C16(Plasm)-18:1 PC, C16(Plasm)-20:4 PE, and C16(Plasm)-22:6 PE, commercially available from Avanti Polar Lipids Inc. of Alabaster, Ala.

phosphatidylglycerols of general formula

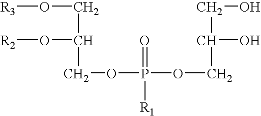

wherein R₁, R₂, and R₃ are defined above. Suitable phosphatidylglycerols suitable for use in the compositions and methods of the invention include, but are not limited to, the 1-acyl-2-acyl-sn-glycero-3-[phospho-rac-(1-glycerol)]s and the 1,2-diacyl-sn-glycero-3-[phospho-rac-(1-glycerol)]s, commercially available from Avanti Polar Lipids Inc. of Alabaster, Ala.

phosphatidylinositols of general formula

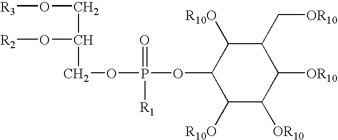

wherein R₁, R₂, R₃, and R₁₀ are defined above. Suitable phosphatidylinositols suitable for use in the compositions and methods of the invention include, but are not limited to, phosphatidylinositol, phosphatidylinositol-4-phosphate, and phosphatidylinositol-4,5-bisphosphate, commercially available from Avanti Polar Lipids Inc. of Alabaster, Ala.

The phospholipids are commercially available or can be obtained by methods well known to those skilled in the art. Representative methods for obtaining phospholipids are described in Sandra Pesch et al., Properties of Unusual Phospholipids Bearing Acetylenic Fatty Acids, Tettrahedron, vol. 15, no. 43, 14,627-14634 (1997); Sepp D. Kohlwein, Phospholipid Synthesis, Sorting, Subcellular Traffic—The Yeast Approach, Trends in Cell Biology, vol. 6, 260-266 (1996), Serguei V. Vinogradov, Synthesis of Phospholipids—Oligodeoxyribonucleotide Conjugates, Tett. Lett., vol. 36, no. 14, 2493-2496 (1995), and references cited therein.

In one embodiment, the phospholipid is Phospholipon®-E:80 (commercially available from Phospholipid GmbH of Cologne, Germany or American Lecithin Company of Oxford, Conn.).

In one embodiment, the phospholipid is Phospholipon®-80G (commercially available from Phospholipid GmbH of Cologne, Germany or American Lecithin Company of Oxford, Conn.).

In one embodiment, the phospholipid is Phospholipon®-85G (commercially available from Phospholipid GmbH of Cologne, Germany or American Lecithin Company of Oxford, Conn.).

In one embodiment, the phospholipid is Phospholipon®-100H (commercially available from Phospholipid GmbH of Cologne, Germany or American Lecithin Company of Oxford, Conn.).

### The Sphingomyelin

Any pharmaceutically acceptable sphingomyelin can be used in the pharmaceutical compositions of the invention.

In one embodiment, the sphingomyelin is

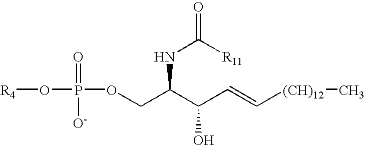

wherein R₁₁ is a C₁-C₂₄ linear, saturated or unsaturated hydrocarbon and R₄ is —CH₂CH₂N(CH₃)₃⁺. In another embodiment, R₁₁ is a C₈-C₂₄ linear, saturated or unsaturated hydrocarbon and R₄ is —CH₂CH₂N(CH₃)₃⁺. In another embodiment, R₁₁ is a C₁₆-C₂₄ linear, saturated or unsaturated hydrocarbon and R₄ is —CH₂CH₂N(CH₃)₃⁺.

Suitable sphingomyelins include, but are not limited to, C2-Sphingomyelin, C6-Sphingomyelin, C18-Sphingomyelin, C6-NBD-Sphingomyelin, and C12-NBD Sphingomyelin, commercially available from Avanti Polar Lipids Inc. of Alabaster, Ala.

Similarly, in another embodiment, the amino acid ester or amide is an ester or amide of lysine and the pharmaceutical composition further comprises a phosphatidyl choline. Without wishing to be bound by theory, it is believed that protonated phosphate groups on the phosphatidyl choline protonates the ε-amine group of lysine to provide a structure as depicted below:

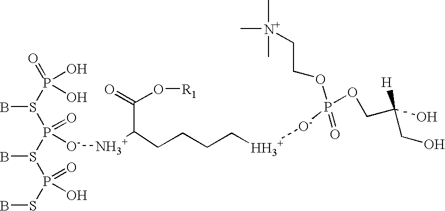

wherein S, B, and R₁ are defined above.

Without wishing to be bound by theory it is also believed that pharmaceutical compositions that comprise an amino acid ester or amide of lysine and further comprise a phospholipid, phosphatidyl choline, or a sphingomyelin that the ester or amide of lysine also forms structures wherein each amino group of the lysine ester or amide is protonated by a phospholipid, phosphatidyl choline, or sphingomyelin molecule. Such a structure is depicted below for a phospholipid:

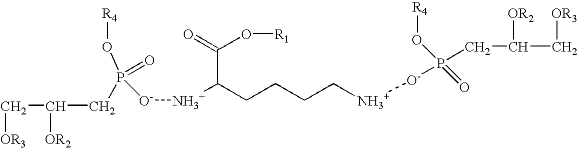

wherein R₁, R₂, R₃, and R₄ are defined above.

The invention also includes pharmaceutical compositions such as those described above that include an ester or amide of lysine, wherein the ester or amide of lysine is replaced with another diamine such as, for example N,N′-dibenzylethylenediamine.

### 7.4.1(i)(c) Pharmaceutical Compositions Comprising Nano-Particles or Micro-Particles Comprising (i) an Amino Acid Ester or Amino Acid Amide and (ii) a Protonated Oligonucleotide Wherein the Amino Acid Ester or Amino Acid Amide is a Diester or Diamide of Aspartic Acid or Glutamic Acid

In another embodiment, the amino acid ester or amide is an ester or amide of aspartic acid or glutamic acid and the side chain carboxylic acid group of the aspartic acid or glutamic acid is also esterified or amidated, i.e., a diester or diamide of aspartic acid or glutamic acid. Without wishing to be bound by theory it is believed that the acidic phosphate groups of the aptamer protonate the amine group of the diester or diamide of aspartic acid or glutamic acid to form a salt between diester or diamide of aspartic acid or glutamic acid and the aptamer as illustrated below for a diester of aspartic acid that is protonated by an oligonucleotide to provide a structure as depicted below:

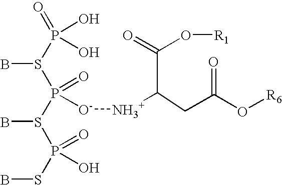

wherein S and B are defined above and R₁ and R₆ are each a C₁-C₂₂ hydrocarbon group.

The diesters of aspartic acid and glutamic acid have the structures:

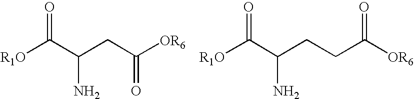

respectively, wherein R₁ and R₆ are defined above. R₁ and R₆ can be the same or different. Typically, however, R₁ and R₆ are the same.

The diamides of aspartic acid and glutamic acid have the structures:

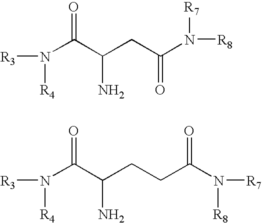

respectively, wherein R₃ and R₄ are defined above (i.e., a hydrogen or C₁-C₂₂ hydrocarbon group), R₇ is the same as R₃, and R₈ is the same as R₄. The amide groups —N(R₃)(R₄) and —N(R₇)(R₈) can be the same or different. Typically, however, the amide groups —N(R₃)(R₄) and —N(R₇)(R₈) are the same.

The molar ratio of acidic groups on the oligonucleotide to the diester or diamide of aspartic acid or glutamic acid typically ranges from about 2:1 to 1:2. In one embodiment, the molar ratio of acidic groups on the aptamer to the diester or diamide of aspartic acid or glutamic acid ranges from about 1.5:1 to 1:1.5. In one embodiment, the molar ratio of acidic groups on the oligonucleotide to the diester or diamide of aspartic acid or glutamic acid ranges from about 1.25:1 to 1:1.25. In one embodiment, the molar ratio of acidic groups on the aptamer to the diester or diamide of aspartic acid or glutamic acid ranges from about 1.1:1. to 1:1.1. In one embodiment, the molar ratio of acidic groups on the oligonucleotide to the diester or diamide of aspartic acid or glutamic acid is about 1:1. A wider range for molar ratio of acidic groups on the oligonucleotide to the diester or diamide of aspartic acid or glutamic acid, however, is also possible. For example, the molar ratio of acidic groups on the oligonucleotide to the diester or diamide of aspartic acid or glutamic acid can range from about 15:1 to 1:15.

### 7.4.2 Pharmaceutical Compositions Comprising Nano-Particles or Micro-Particles Comprising (i) an Oligonucleotide, (ii) a Divalent Metal Cation, and (iii) Optionally a Carboxylate, a Phospholipid, a Phosphatidyl Choline, or a Sphingomyelin

In another embodiment, the pharmaceutical compositions comprise nano-particles or micro-particles comprising (i) an oligonucleotide, (ii) a divalent metal cation and (iii) optionally a carboxylate, a phospholipid, a phosphatidyl choline, or a sphingomyelin. In one embodiment, the particles are nano-particles. In one embodiment, the particles are micro-particles. Without wishing to be bound by theory, it is believed that the divalent metal cation interacts with the phosphate groups on the oligonucleotide to form a structure as depicted below:

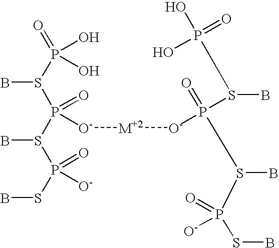

wherein M⁺² is a divalent metal cation and B and S are defined above.

Without wishing to be bound by theory, it is believed that when the pharmaceutical composition includes the optional carboxylate, phospholipid, phosphatidyl choline, or sphingomyelin the divalent metal cation interacts with the phosphate groups on the aptamer and the carboxylate, phospholipid, phosphatidyl choline, or sphingomyelin to form a structure as depicted below for a carboxylate:

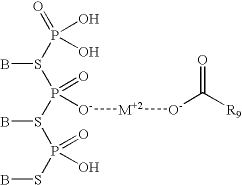

wherein M⁺², B, S are defined above and R₉ is a C₁-C₂₁ hydrocarbon. Without wishing to be bound by theory, it is believed that the structures are similar to the structures formed between an aptamer; the amino acid lysine; and a carboxylic acid, a phospholipid, phosphatidyl choline, or a sphingomyelin, described above, except that the divalent metal cation replaces the lysine.

Without wishing to be bound by theory it is also believed that when the pharmaceutical composition includes the optional carboxylate, phospholipid, phosphatidyl choline, or sphingomyelin the divalent metal cation can also interact with more than one carboxylate, phospholipid, phosphatidyl choline, or sphingomyelin to form a structure as depicted below for a carboxylate:

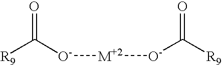

wherein M⁺² and R₉ are defined above.

In one embodiment, the pharmaceutical composition comprises a carboxylate.

In one embodiment, the pharmaceutical composition comprises a phospholipid.

In one embodiment, the pharmaceutical composition comprises phosphatidyl choline.

In one embodiment, the pharmaceutical composition comprises a sphingomyelin.

Any of the oligonucleotide described above can be used in the pharmaceutical compositions.

The carboxylate can be obtained from any pharmaceutically acceptable carboxylic acid. Any of the carboxylic acids described herein can be used to provide the carboxylate.

In one embodiment, the carboxylic acid is an N-acyl amino acid of general formula (III). Any N-acyl amino acid of general formula (III) described above can be used in the pharmaceutical compositions.

Any of the phospholipids described above can be used in the pharmaceutical compositions.

Any of the sphingomyelins described above can be used in the pharmaceutical compositions.

Suitable divalent metal cations include, but are not limited to, the alkaline earth metal cations, Mg⁺², Zn⁺², Cu⁺², and Fe⁺². Preferred divalent metal cations are Ca⁺², Mg⁺², Zn^{÷2}, Cu⁺², and Fe⁺².

The combined molar ratio of anionic groups on the oligonucleotide and anionic groups on the carboxylate, phospholipid, phosphatidyl choline, or sphingomyelin to the divalent metal cation typically ranges from about 4:1 to 1:4. In one embodiment, the combined molar ratio of anionic groups on the oligonucleotide and anionic groups on the carboxylate, phospholipid, phosphatidyl choline, or sphingomyelin to the divalent metal cation ranges from about 3:1 to 1:3. In one embodiment, the combined molar ratio of anionic groups on the oligonucleotide and anionic groups on the carboxylate, phospholipid, phosphatidyl choline, or sphingomyelin to the divalent metal cation ranges from about 2.5:1 to 1:2.5. In one embodiment, the combined molar ratio of anionic groups on the oligonucleotide and anionic groups on the carboxylate, phospholipid, phosphatidyl choline, or sphingomyelin to the divalent metal cation ranges from about 2:1. to 1:2. In one embodiment, the combined molar ratio of anionic groups on the oligonucleotide and anionic groups on the carboxylate, phospholipid, phosphatidyl choline, or sphingomyelin to the divalent metal cation is about 2:1. A wider range for the molar ratio of anionic groups on the oligonucleotide and anionic groups on the carboxylate, phospholipid, phosphatidyl choline, or sphingomyelin to the divalent metal cation, however, is also possible. For example, the molar ratio of anionic groups on the oligonucleotide and anionic groups on the carboxylate, phospholipid, phosphatidyl choline, or sphingomyelin to the divalent metal cation can range from about 20:1 to 1:20. In one embodiment, the molar ratio of anionic groups on the oligonucleotide and anionic groups on the carboxylate, phospholipid, phosphatidyl choline, or sphingomyelin to the divalent metal cation ranges from about 15:1 to 1:15. In one embodiment, the molar ratio of anionic groups on the oligonucleotide and anionic groups on the carboxylate, phospholipid, phosphatidyl choline, or sphingomyelin to the divalent metal cation ranges from about 10:1 to 1:10. In one embodiment, the molar ratio of anionic groups on the oligonucleotide and anionic groups on the carboxylate, phospholipid, phosphatidyl choline, or sphingomyelin to the divalent metal cation ranges from about 5:1 to 1:5.

### 7.4.3 General Characteristics of the Pharmaceutical Compositions

As described above, the pharmaceutical compositions comprise nano-particles or micro-particles of an oligonucleotide and a pharmaceutically acceptable organic base or comprises nano-particles or micro-particles of an oligonucleotide and a divalent metal cation. The nano-particles or micro-particles can be readily dispersed in a pharmaceutically acceptable solvent to provide a composition that is injectable. Accordingly, in one embodiment, the pharmaceutical composition comprises

(a) nano-particles or micro-particles comprising (i) an oligonucleotide and (ii) a pharmaceutically acceptable organic base or a divalent metal ion, and

(b) a pharmaceutically acceptable solvent.

The nano-particles or micro-particles are dispersed in the pharmaceutically acceptable solvent.

In one embodiment, the particles are nano-particles.

In one embodiment, the particles are micro-particles.

The nano-particle or micro-particles can be dispersed in a pharmaceutically acceptable solvent by adding the pharmaceutically acceptable solvent to the nano-particles or micro-particles with agitation or shaking.

The resulting dispersion of nano-particles or micro-particles in a solvent are injectable and can be administered to an animal, for example, subcutaneously or intravenously. The resulting dispersion of nano-particles in a solvent can also be sterile filtered to provide sterile compositions.

In one embodiment, the concentration of the oligonucleotide dispersed in the solvent is greater than about 2 percent by weight of the pharmaceutical composition. In one embodiment, the concentration of the oligonucleotide dispersed in the solvent is greater than about 5 percent by weight of the pharmaceutical composition. In one embodiment, the concentration of the oligonucleotide dispersed in the solvent is greater than about 7.5 percent by weight of the pharmaceutical composition. In one embodiment, the concentration of the oligonucleotide dispersed in the solvent is greater than about 10 percent by weight of the pharmaceutical composition. In one embodiment, the concentration of the oligonucleotide dispersed in the solvent is greater than about 12 percent by weight of the pharmaceutical composition. In one embodiment, the concentration of the oligonucleotide dispersed in the solvent is greater than about 15 percent by weight of the pharmaceutical composition. In one embodiment, the concentration of the oligonucleotide dispersed in the solvent ranges from about 2 percent to 5 percent by weight of the pharmaceutical composition. In one embodiment, the concentration of the oligonucleotide dispersed in the solvent ranges from about 2 percent to 7.5 percent by weight of the pharmaceutical composition. In one embodiment, the concentration of the oligonucleotide dispersed in the solvent ranges from about 2 percent to 10 percent by weight of the pharmaceutical composition. In one embodiment, the concentration of the oligonucleotide dispersed in the solvent ranges from about 2 percent to 12 percent by weight of the pharmaceutical composition. In one embodiment, the concentration of the oligonucleotide dispersed in the solvent ranges from about 2 percent to 15 percent by weight of the pharmaceutical composition. In one embodiment, the concentration of the oligonucleotide dispersed in the solvent ranges from about 2 percent to 20 percent by weight of the pharmaceutical composition.

In one embodiment, the pharmaceutically acceptable organic solvent is a solvent that is recognized as GRAS by the FDA for administration or consumption by animals.

In one embodiment, the pharmaceutically acceptable organic solvent is a solvent that is recognized as GRAS by the FDA for administration or consumption by humans.

In one embodiment, the pharmaceutically acceptable solvent is water.

In one embodiment, the pharmaceutically acceptable solvent is an organic solvent.

Many oligonucleotide containing pharmaceutical compositions require the inclusion of a surfactant (i.e., a compound that reduces the surface tension of a liquid), in particular a cationic surfactant, so as to provide a composition that can be formulated with a solvent to provide a liquid composition that has a sufficiently high concentration of the oligonucleotide. Surfactants, however, can be toxic. An advantage of the pharmaceutical compositions of the invention is that, unlike prior art oligonucleotide containing pharmaceutical compositions, they do not require the inclusion of a surfactant. In one embodiment, the pharmaceutical compositions of the invention are substantially free of a cationic surfactant. In one embodiment, the pharmaceutical compositions of the invention are substantially free of a surfactant.

Without wishing to be bound by theory it is believed that the oligonucleotide and the pharmaceutically acceptable organic base or the oligonucleotide and the divalent metal ion of the nano-particles or micro-particles interact ionically to form a salt, i.e., there is no covalent bonding between the oligonucleotide and the pharmaceutically acceptable organic base or the oligonucleotide and the divalent metal ion.

In one embodiment, the nano-particles or micro-particles are multi-valent, i.e., there is more than one pharmaceutically acceptable organic base or divalent metal ion associated with each oligonucleotide.

The pharmaceutical compositions, when in the form of nano-particles, provides a formulation that enables intracellular delivery of the oligonucleotide. Without wishing to be bound by theory it is believed that intracellular delivery of the oligonucleotide is facilitated due to both the nano-particle size of the composition and the components of the pharmaceutical composition (i.e., the oligonucleotide associated with a pharmaceutically acceptable organic base or divalent metal ion).

Pharmaceutical compositions comprising nano-particles and further comprising a solvent are advantageous because the nano-particle containing compositions can be sterile filtered, i.e., filtered through a 0.22 μm filter, to provide a sterile solution.

### 7.4.4 Optional Additives

The pharmaceutical compositions can optionally comprise one or more additional excipients or additives to provide a dosage form suitable for administration to an animal. When administered to an animal, the oligonucleotide containing pharmaceutical compositions are typically administered as a component of a composition that comprises a pharmaceutically acceptable carrier or excipient so as to provide the form for proper administration to the animal. Suitable pharmaceutical excipients are described in Remington's Pharmaceutical Sciences 1447-1676 (Alfonso R. Gennaro ed., 19th ed. 1995), incorporated herein by reference. The pharmaceutical compositions can take the form of solutions, suspensions, emulsion, tablets, pills, pellets, capsules, capsules containing liquids, powders, suppositories, emulsions, aerosols, sprays, suspensions, or any other form suitable for use.

In one embodiment, the pharmaceutical compositions are formulated for intravenous or parenteral administration. Typically, compositions for intravenous or parenteral administration comprise a suitable sterile solvent, which may be, for example, an isotonic aqueous buffer. Compositions for injection can optionally include a local anesthetic such as lidocaine to lessen pain at the site of the injection. Generally, a pharmaceutical composition for administration by injection is obtained by dispersing the solid nano-particles or micro-particles in the pharmaceutically acceptable solvent by adding the solvent to the solid nano-particles or micro-particles with shaking to provide a suspension of the nano-particles or micro-particles in the solvent that is suitable for administration by injection. For example, the solid nano-particles or micro-particles can be supplied as a dry lyophilized powder or water free concentrate in a hermetically sealed container, such as an ampoule or sachette, indicating the quantity of active agent. Where oligonucleotide containing pharmaceutical compositions are to be administered by infusion, they can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

In another embodiment, the pharmaceutical compositions are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Oral compositions can include standard excipients such as mannitol, lactose, starch, magnesium stearate, sodium saccharin, cellulose, and magnesium carbonate. Typically, the excipients are of pharmaceutical grade. Orally administered compositions can also contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. A time-delay material such as glycerol monostearate or glycerol stearate can also be used.

### 7.4.5 Methods of Preparing the Oligonucleotide Containing Pharmaceutical Compositions

The pharmaceutical compositions can be prepared by dissolving an inorganic salt of the oligonucleotide, typically a potassium or sodium salt, in a solvent in which it is soluble, for example methanol or water, and adjusting the pH of the resulting solution to a value of between about 2 and 3 with an organic acid, such as formic acid, as depicted below for an aptamer:

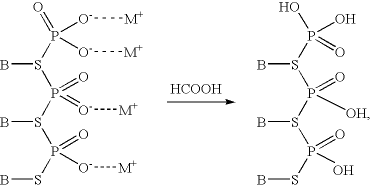

wherein S and B are defined above and M⁺ is a metal ion, to provide a solution of the protonated oligonucleotide.

The resulting solution of protonated oligonucleotide is then dialyzed against water to remove excess formic acid and formate salts and if, for example, the neutralization is conducted in a methanol solvent, to replace the methanol with water. The water can then be removed from the aqueous solution of the protonated oligonucleotide by lyophilization to provide the protonated oligonucleotide or, alternatively, the aqueous solution of the protonated oligonucleotide can be dialyzed against methanol to replace the water with methanol and then simply removing the methanol under reduced pressure to provide the protonated oligonucleotide.

A solution of the protonated oligonucleotide can also be prepared using a cation exchange resin. Any cationion exchange resin known to one skilled in the art can be used, for example, a Strata® SCX cation exchange resin (commercially available from Phenomenex of Torrance, Calif.) or a DOWEX® cation exchange resin, such as DOWEX® 50 (commercially available from Dow Chemical Company of Midland, Mich.) can be used. Typically, a column containing the cation exchange resin is first washed with an acidic solution to protonate the resin and then a solution of the inorganic salt of the oligonucleotide, typically a potassium or sodium salt, in a solvent, for example methanol or water, is passed through the resin to provide, as the eluant, a solution of the protonated oligonucleotide.

To prepare the pharmaceutical compositions comprising a protonated oligonucleotide and an a pharmaceutically acceptable organic base (using an amino acid ester or amide as a representative pharmaceutically acceptable organic base), the protonated oligonucleotide is dissolved in a solvent, such as methanol, typically with stirring, and to the resulting solution is then added the amino acid ester or amide, as depicted below:

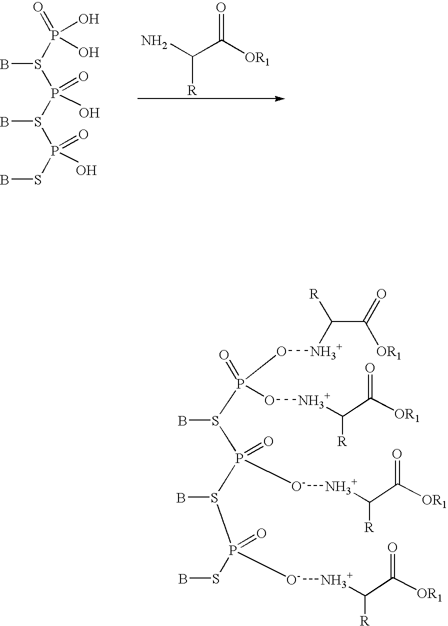

wherein S, B, R, and R₁ are defined above.

Any other components of the pharmaceutical composition, such as a carboxylic acid, phospholipid, phosphatidyl choline, sphingomyelin, or diester or diamide of aspartic or glutamic acid are then added to the resulting solution.

Typically, sufficient amino acid ester or amide, and any other components, are added to provide a solution having a pH value ranging from about 5 to 9. In one embodiment, sufficient amino acid ester or amide, and any other components, are added to provide a solution having a pH value ranging from about 6 to 8. In one embodiment, sufficient amino acid ester or amide, and any other components, are added to provide a solution having a pH value of about 7. The pH can be readily measured by removing a few microliters of the solution and applying it to a wet pH test strip (such as commercially available from Sigma-Aldrich of Milwaukee, Wis.) that indicates the pH of the solution by the color of the test strip after the solution is applied. The solvent is then removed under reduced pressure to provide a composition comprising the amino acid ester or amino acid amide and the oligonucleotide. In another embodiment, an anti-solvent (for example, water) is added to the solution to precipitate a composition comprising the amino acid ester or amino acid amide and the oligonucleotide.

Nano-particles of the composition comprising the amino acid ester or amino acid amide and the oligonucleotide are then formed using methods known to those skilled in the art for making nano-particles. Suitable methods for forming nano-particles include, but are not limited to, the following methods:

Mechanical Methods:

Spray drying (See, A. Gomez et al., J. Aerosol Sci, 29 (1998) 561-74 and Y. Yoon, et al., J. Controlled Rel., 100 (2004), 379).

Milling (See, S. Buchmann, et al., Proceedings of the 42nd Annual Congress of the International Association for Pharmaceutical Technology (APV) Mainz, (1996) 124).

Sonication and high speed mixing (See, T. Eldem, et al., Pharm. Res. 8 (1991) 47-54; EP 0167825; J. X. Wang, et al, Eur. J. Pharm. Biopharm., 54 (2002) 285-290; and D. Z. Hou, et al., Biomaterials, 24 (2003) 1781-1785).

High pressure homogenization (See, U.S. Pat. No. 5,858,410; PCT/EP00/06535, and M. Radtke, New Drugs, 3 (2001) 62-68).

Direct mixing (See, X. Gao and L. Huang, Biochemistry 35 (1996) 1027-1036; J. Dileo, et al., Molec. Ther., 7 (2003) 640-648; R. E. Eliaz and F. C. Szoka Jr., Gene Ther. 9 (2002) 1230-1237; N. Shi, et al., Proc. Natl. Acad. Sci. USA, 98 (2001) 12754-12759; Y. Zhang, et al., Molec. Ther., 6 (2002) 67-72; and Y. Zhang, et al., J. Gene Med., 5 (2003) 1039-1045.

Chemical or Solution Based Methods:

Precipitation (See, GB 2200048; P. Gassmann, et al., Eur. J. Pharm. Biopharm., 40 (1994) 64-72; and N. Rasenack, et al., Pharm. Res. 19 (2002) 1894-1900).

Microemulsion (See, U.S. Pat. No. 5,250,236; R. Cavalli, et al., Eur. J. Pharm. Biopharm., 43 (1996) 110-115; M. R. Gasco, Pharm. Technol. Eur., 9 (1997) 52-58; R. Cortesi, et al., Biomaterials, 23 (2002) 2283-2294; R. Cavalli, et al., Pharmazie, 53 (1998) 392-396; and M. Igartua, et al., Int. J. Pharm., 233 (2002) 149-157).

Solvent emulsification-evaporation or diffusion (See, B. Sjostrom, et al., Pharm. Res., 12 (1995) 39-48; P. Shahgaldian, et al., Int. J. Pharm., 253 (2003) 23-38; A. Dubes, et al., Eur. J. Pharm. Biopharm., 55 (2003) 279-282; M. Trotta, et al., Int. J. Pharm., 257 (2003) 153-160; and F. Q. Hu, et al., Int. J. Pharm., 239 (2002) 121-128).

W/O/W double emulsion (See, R. Cortesi, et al., Biomaterials, 23 (2002) 2283-2294).

Direct mixing (See, X. Gao and L. Huang, Biochemistry, 35 (1996) 1027-1036; J. Dileo, et al., Molec. Ther., 7 (2003) 640-648; R. E. Eliaz and F. C. Szoka Jr., Gene Ther., 9 (2002) 1230-1237; N. Shi, et al., Proc. Natl. Acad. Sci. USA, 98 (2001) 12754-12759; Y. Zhang, et al., Molec. Ther., 6 (2002) 67-72; Y. Zhang, et al., J. Gene Med., 5 (2003) 1039-1045; H. E. Hofland, et al., Proc. Natl. Acad. Sci. USA, 93 (1996) 7305-7309).

Detergent dialysis (See, H. E. Hofland, et al., Proc. Natl. Acad. Sci. USA, 93 (1996) 7305-7309 and C. Y. Wang and L. Huang, Proc. Natl. Acad. Sci. USA, 84 (1987) 7851-7855).

Ethanol dialysis (See, S. Batzri and E. D. Korn, Biochim. Biophys. Acta, 298 (1973) 1015-1019; M. J. Campbell, Biotechniques, 18 (1995) 1027-1032; P. G. Arscott, et al., Biopolymers, 36 (1995) 345-364; J. Piskur and A. Rupprecht, FEBS Lett., 375 (1995) 174-178; A. L. Bailey and S. M. Sullivan, Biochim. Biophys. Acta, 1468 (2000) 239-252; N. Maurer, et al., Biophys. J., 80 (2001) 2310-2326; D. V. Morrissey, et al., Nat. Biotechnol., 23 (2005) 1002-1007; A. D. Judge, et al., Molec. Ther., 13 (2006) 494-505; T. S. Zimmermann, et al., Nature, 441 (2006) 111-114; and W. Li and F. C. Szoka, “Bioresponsive targeted charge neutral lipid vesicles for systemic gene delivery” in Gene Transfer: Delivery and Expression of DNA and RNA, Cold Spring Harbor Laboratory Press, New York, edited by T. Friedmann and J. Rossi (2006) 441-450).

Molded fabrication (See, J. P. Rolland, et al., J. Am. Chem. Soc., 127 (2005) 10096-10100).

Similar methods can be used to prepare micro-particles. One of ordinary skill in the art would readily know how to obtain nano-particles or micro-particles.

To prepare the pharmaceutical compositions comprising an oligonucleotide; a divalent metal cation; and, optionally, a carboxylate, a phospholipid, a phosphatidyl choline, or a sphingomyelin, the protonated oligonucleotide is dissolved in a solvent, such as methanol, and to the resulting solution is added a metal salt, such as a metal acetate, or a metal hydroxide, preferably with stirring. To the resulting solution is then added, if desired, the carboxylic acid, phospholipid, phosphatidyl choline, or sphingomyelin, preferably with stirring. The solvent is then removed under reduced pressure to provide a composition comprising the oligonucleotide; a divalent metal cation; and, optionally, a carboxylate, a phospholipid, a phosphatidyl choline, or a sphingomyelin. In another embodiment, an anti-solvent (for example, water) is added to the solution to precipitate a composition comprising the oligonucleotide; a divalent metal cation; and, optionally, a carboxylate, a phospholipid, a phosphatidyl choline, or a sphingomyelin. Nano-particles or micro-particles of the resulting composition comprising the oligonucleotide; a divalent metal cation; and, optionally, a carboxylate, a phospholipid, a phosphatidyl choline, or a sphingomyelin are then formed using methods known to those skilled in the art for making nano-particles or micro-particles, including, but not limited to, those described above.

To prepare the pharmaceutical compositions comprising a protonated oligonucleotide and polylysine, a polylysine solution (such as a methanol solution) is slowly added to a solution (such as a methanol solution) of the protonated oligonucleotide, preferably with stirring, and the pH of the resulting solution monitored to provide a solution having the desired pH value. The methanol is then removed under reduced pressure to provide a composition comprising a protonated oligonucleotide and polylysine. In another embodiment, an anti-solvent (for example, water) is added to the solution to precipitate a composition comprising the oligonucleotide and polylysine. Nano-particles or micro-particles of the resulting composition comprising a protonated oligonucleotide and polylysine are then formed using methods known to those skilled in the art for making nano-particles or micro-particles, including, but not limited to, those described above.

The polylysine is obtained from commercially available polylysine hydrobromide (commercially available from Sigma-Aldrich, St. Louis, Mo.) by simply neutralizing a solution (such as a methanol or water solution) of the polylysine hydrobromide with ammonium hydroxide to provide a solution having a pH value ranging from about 10 to 12. The resulting solution of polylysine is then dialyzed against water to remove excess ammonium bromide and ammonium hydroxide and if, for example, the neutralization is conducted in a methanol solvent, to replace the methanol with water. The water can then be removed from the aqueous solution of the polylysine by lyophilization to provide the polylysine or, alternatively, the aqueous solution of the polylysine can be dialyzed against methanol to replace the water with methanol and then the methanol simply removed under reduced pressure to provide the polylysine.

### 7.5 Methods of Treating a Condition in an Animal

The pharmaceutical compositions of the invention provide a convenient method for administering oligonucleotides to an animal. As a result, the pharmaceutical compositions of the invention are useful in human medicine and veterinary medicine. Accordingly, the invention further relates to a method of treating or preventing a condition in an animal comprising administering to the animal an effective amount of the pharmaceutical composition of the invention.

The pharmaceutical compositions of the invention comprising nano-particles are particularly useful when intracellular delivery of the oligonucleotide is desired. As described above, the nano-particle pharmaceutical compositions of the invention facilitate intracellular delivery of the oligonucleotide.

In one embodiment, the invention relates to methods of treating a condition in an animal comprising administering to an animal in need thereof an effective amount of a pharmaceutical composition of the invention.

In one embodiment, the invention relates to methods of preventing a condition in an animal comprising administering to an animal in need thereof an effective amount of a pharmaceutical composition of the invention.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical. The mode of administration is left to the discretion of the practitioner. In most instances, administration will result in the release of the oligonocleotide into the bloodstream.

In one embodiment, the method of treating or preventing a condition in an animal comprises administering to the animal in need thereof an effective amount of an oligonucleotide by parenterally administering the pharmaceutical composition of the invention. In one embodiment, the pharmaceutical compositions are administered by infusion or bolus injection. In one embodiment, the pharmaceutical composition is administered subcutaneously. In one embodiment, the pharmaceutical composition is administered intravenously.

In one embodiment, the method of treating or preventing a condition in an animal comprises administering to the animal in need thereof an effective amount of an oligonucleotide by orally administering the pharmaceutical composition of the invention. In one embodiment, the composition is in the form of a capsule or tablet.

The pharmaceutical compositions can also be administered by any other convenient route, for example, topically, by absorption through epithelial or mucocutaneous linings (e.g., oral, rectal, and intestinal mucosa, etc.).

The pharmaceutical compositions can be administered together with another biologically active agent.

In one embodiment, the animal is a mammal.

In one embodiment the animal is a human.

In one embodiment, the animal is a non-human animal.

In one embodiment, the animal is a canine, a feline, an equine, a bovine, an ovine, or a porcine.

The effective amount administered to the animal depends on a variety of factors including, but not limited to the type of animal being treated, the condition being treated, the severity of the condition, and the specific oligonucleotide being administered. One of ordinary skill in the art will readily know what is an effective amount of the pharmaceutical composition to treat a condition in an animal.

In one embodiment, the oligonucleotide is a anti-Vascular Endothelial Growth Factor (VEGF) aptamer. In one embodiment, the oligonucleotide is a anti-Vascular Endothelial Growth Factor (VEGF) aptamer and the disorder is an ocular disorder. Representative ocular disorders include, but are not limited to, age-related macular degeneration, optic disc neovascularization, iris neovascularization, retinal neovascularization, choroidal neovascularization, corneal neovascularization, vitreal neovascularization, glaucoma, pannus, pterygium, macular edema, vascular retinopathy, retinal degeneration, uveitis, inflammatory diseases of the retina, or proliferative vitreoretinopathy. Virtually any method of delivering a medication to the eye may be used for the delivery of the pharmaceutical compositions of the invention. In one embodiment, the pharmaceutical composition is administered intravitreally, for example, via intravitreal injection. In one embodiment, the pharmaceutical composition is administered transclerally.

In one embodiment, the oligonucleotide is an oligonucleotide that inhibits angiogenesis.

In one embodiment, the oligonucleotide is an oligonucleotide that inhibits angiogenesis and the disease being treated is cancer. In one embodiment, the oligonucleotide is an oligonucleotide that inhibits angiogenesis and the disease being treated is a solid tumor.

### 7.6 Kits

The invention encompasses kits that can simplify the administration of the pharmaceutical composition to an animal. A typical kit of the invention comprises a unit dosage form of a pharmaceutical composition of the invention. In one embodiment, the unit dosage form is a container, such as a vial, which can be sterile, containing a pharmaceutical composition of the invention. The kit can further comprise a label or printed instructions instructing the use of the pharmaceutically active compound to treat a condition. In another embodiment, the kit comprises a unit dosage form of a pharmaceutical composition of the invention and a syringe for administering the pharmaceutical composition.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 8. Examples

### Example 1

### Preparation of Amino Acid Esters and Amino Acid-Vitamin Esters

### Amino Acid Esters

Tryptophane butanoate: 1 g of tryptophane butanoate hydrochloride salt (commercially available from Sigma-Aldrich, St. Louis, Mo.) was suspended in 25 mL of dichloromethane and 600 μl of triethylamine was added to the suspension with stirring. Stirring was continued for 15 min and the resulting solution was transferred to a separatory funnel. The organic solution was washed twice with 25 mL of water followed by 25 mL of saturated aqueous sodium bicarbonate. The organic layer was then dried over anhydrous sodium sulfate and concentrated under reduced pressure to provide tryptophane butanoate. The structure was confirmed using mass spectroscopy.

Tryptophane octanoate: 4 g of tryptophane butanoate hydrochloride salt (commercially available from Sigma-Aldrich, St. Louis, Mo. (www.sima-aldrich.com)) was suspended in 100 mL of dichloromethane and 3 ml of triethylamine was added to the suspension with stirring. Stirring was continued for 15 min and the resulting solution was transferred to a separatory funnel. The organic solution was washed twice with 25 mL of water followed by 25 mL of saturated aqueous sodium bicarbonate. The organic layer was then dried over anhydrous sodium sulfate and concentrated under reduced pressure to provide tryptophane octanoate. The structure was confirmed using mass spectroscopy.

Tyrosine butanoate: 18.19 g of tyrosine was suspended in a solution of 9.8 g of concentrated sulfuric acid, 40 mL water, 40 mL of butanol, and 200 mL of toluene in a 500 mL round bottom flask equipped with a condenser and a Dean-Stark apparatus. The resulting solution was heated at reflux temperature until no more water could be distilled. The resulting solution was cooled in an ice bath, which caused the solution to separate into two phases. The upper phase was discarded and the lower phase, an oily syrup, was retained. The syrup was mixed with sufficient 5% aqueous sodium bicarbonate solution to neutralize acidic impurities to provide a solid that was collected by filtration and washed with cold water. The resulting solid was re-crystallized in ethyl acetate.

Isoleucine butyrate: 26.23 g of isoleucine was dissolved in a solution of 20 g of concentrated sulfuric acid, 20 mL water, 40 mL of butanol, and 200 mL of toluene in a 500 mL round bottom flask equipped with a condenser and a Dean-Stark apparatus. The resulting solution was heated at reflux temperature until no more water could be distilled. The resulting solution was then cooled to room temperature and washed with saturated aqueous sodium bicarbonate to neutralize acidic impurities, washed with saturated brine, and dried over anhydrous sodium sulfate. The solvent was removed under reduced pressure and the resulting liquid distilled under vacuum to provide isoleucine butyrate as a colorless liquid.

Phenylalanine butyrate: 16.52 g of isoleucine was dissolved in a solution of 10 g of concentrated sulfuric acid, 20 mL water, 20 mL of butanol, and 200 mL of toluene in a 500 mL round bottom flask equipped with a condenser and a Dean-Stark apparatus. The resulting solution was heated at reflux temperature until no more water could be distilled. The resulting solution was then cooled to room temperature and washed with saturated aqueous sodium bicarbonate to neutralize acidic impurities, washed with saturated brine, and dried over anhydrous sodium sulfate. The solvent was removed under reduced pressure and the resulting liquid distilled under vacuum to provide phenylalanine butyrate.

Phenylalanine octanoate: 16.52 g of phenylalanine was dissolved in a solution of 10 g of concentrated sulfuric acid, 20 mL water, 20 mL of octanol, and 120 mL of toluene in a 500 mL round bottom flask equipped with a condenser and a Dean-Stark apparatus. The resulting solution was heated at reflux temperature until no more water could be distilled. The resulting solution was then cooled to room temperature and washed with saturated aqueous sodium bicarbonate to neutralize acidic impurities, washed with saturated brine, and dried over anhydrous sodium sulfate. The solvent was then removed under reduced pressure to provide phenylalanine octanoate as a white solid that was purified using a silica gel column eluted with a 1:9 methanol:dichloromethane mixture.

Phenylalanine dodecanoate: 16.52 g of phenylalanine was dissolved in a solution of 10 g of concentrated sulfuric acid, 20 mL water, 20 mL of dodecanol, and 120 mL of toluene in a 500 mL round bottom flask equipped with a condenser and a Dean-Stark apparatus. The resulting solution was heated at reflux temperature until no more water could be distilled. The resulting solution was then cooled to room temperature and washed with saturated aqueous sodium bicarbonate to neutralize acidic impurities, washed with saturated brine, and dried over anhydrous sodium sulfate. The solvent was then removed under reduced pressure to provide phenylalanine dodecanoate as a solid that was purified using a silica gel column eluted with a 1:9 methanol:dichloromethane mixture.

Tyrosine octanoate: 9.06 g of tyrosine was dissolved in a solution of 10 g of concentrated sulfuric acid, 20 mL water, 10 mL of octanol, and 200 mL of toluene in a 500 mL round bottom flask equipped with a condenser and a Dean-Stark apparatus. The resulting solution was heated at reflux temperature until no more water could be distilled. The resulting solution was then cooled to room temperature and washed with saturated aqueous sodium bicarbonate to neutralize acidic impurities to provide an emulsion. About 150 mL of ethyl acetate was added to the emulsion to provide two phases. The aqueous phase was discarded and the organic phase washed with saturated Brine and dried over anhydrous sodium sulfate. The solvent was the removed under reduced pressure to provide tyrosine octanoate as a white solid that was purified using a silica gel column eluted with a 1:9 methanol:dichloromethane mixture.

Isoleucine octanoate: 13.1 g of isoleucine was dissolved in a solution of 10 g of concentrated sulfuric acid, 20 mL water, 20 mL of octanol, and 200 mL of toluene in a 500 mL round bottom flask equipped with a condenser and a Dean-Stark apparatus placed in an oil bath. The resulting solution was heated at reflux temperature until no more water could be distilled. The resulting solution was then cooled to room temperature, diluted with 120 mL of ethyl acetate and the organic layer washed with saturated aqueous sodium bicarbonate to neutralize acidic impurities, washed with saturated Brine, and dried over anhydrous sodium sulfate. The solvent was removed under reduced pressure and the resulting liquid distilled to provide isoleucine octanoate as a colorless liquid.

Proline butanoate: 34.5 g of proline was suspended in a solution of 35 g of concentrated sulfuric acid, 40 mL water, 120 mL of butanol, and 200 mL of toluene in a 500 mL round bottom flask equipped with a condenser and a Dean-Stark apparatus. The resulting solution was heated at reflux temperature until no more water could be distilled. The resulting solution was then cooled to room temperature, washed with saturated aqueous sodium bicarbonate to neutralize acidic impurities, washed with saturated Brine, and dried over anhydrous sodium sulfate. The solvent was removed under reduced pressure and the resulting liquid distilled to provide proline butanoate as a colorless liquid.

Lysine hexadecanoate: BOC protected lysine (6.25 g, 0.018 mole) was dissolved in about 40 mL of tetrahydrofuran under a nitrogen atmosphere. The solution was cooled to about 0° C. using an ice-water bath and carbonyl diimidazole (2.93 g, 0.018 mole) was added to the cooled solution. The reaction mixture was then allowed to stir for about 5 min. at about 5° C. and then for about 30 min. at room temperature. To the resulting solution was then added by dropwise addition a solution of hexadecanol (4.38 g, 0.018 mole) in about 10 mL of tetrahydrofuran. The resulting solution was then warmed to about 45° C. and allowed to stir for about 12 h. After stirring, the solvent was evaporated under reduced pressure; the resulting residue dissolved in ethyl acetate; the ethyl acetate washed with 0.1 N hydrochloric acid (3 times), saturated aqueous sodium hydrogen carbonate (3 times), and brine (3 times); and the organic phase dried (Na₂SO₄). The ethyl acetate was then removed under reduced pressure to provide crude BOC protected lysine hexadecanoate that was purified using silica gel column chromatography eluted with 0 to 20 percent ethyl acetate in hexane. The solvent was then evaporated under reduced pressure to provide purified BOC protected lysine hexadecanoate. Trifluoroacetic acid (20 mL) was added to the purified BOC protected lysine hexadecanoate and the resulting reaction mixture stirred for about 5 h. Excess trifluoroacetic acid was removed under reduced pressure. The resulting residue was then dissolved in methanol and passed through a Dowex 550A(OH) resin (50 g) (commercially available from Dow Chemical Company of Midland Mich.) and the solvent removed under reduced pressure to provide lysine hexadecanoate that was dried under vacuum to provide dried lysine hexadecanoate (3.6 g).

### Amino Acid-Vitamin Esters

Esters of naturally occurring vitamin and amino acid are synthesized as follows. A BOC-protected amino acid (30.7 mmol) is dissolved in anhydrous tetrahydrofuran (200 mL) under an argon atmosphere, the mixture cooled to 4° C. in an ice bath, and activated by adding carbonyldiimidazole (5 g, 30.1 mmol). The resulting reaction mixture is then warmed to room temperature and allowed to further react for 1 hour. A vitamin containing a hydroxyl group (for example, vitamin E or vitamin A) is then added to the mixture and the mixture heated to 50° C. After 24 hours the reaction mixture is cooled to room temperature and the tetrahydrofuran removed under reduced pressure. The resulting oil is dissolved in ethyl acetate and extracted twice with 0.25M HCl and the organic layer is dried using sodium sulfate and evaporated to dryness. Further purification is achieved using column chromatography with a silica gel solid support and eluting with 20% methyl tert-butyl ether/hexane. The resulting yield is approximately 16.3 mmol of BOC-protected amino acid vitamin ester (m/z 761.0, when the vitamin is α-tocopherol and the amino acid is lysine).

Purified vitamin-amino acid ester salts with trifluoroacetic acid are obtained by stirring the vitamin-amino acid ester in 30% trifluoroacetic/dichloromethane (50 mL) for 2 hours. Dichloromethane and excess trifluoroacetic acid are then removed under reduced pressure and the salt dissolved in fresh dichloromethane (200 mL). DOWEX anion exchange resin (Sigma Aldrich St. Louis Miss.) (200 mL, 200 mmol pyridinium ion) is then added and the resulting mixture stirred for 30 minutes and filtered to provide the free base of the vitamin-amino acid ester. Further purification is achieved by loading the free base onto a tosic acid functionalized silica gel (commercially available from Siliscycle, Inc. of Quebec, Canada), (27.5 g, 1.2 eq), washing with dichloromethane, and eluting with equivalents of triethlyamine in dichloromethane. Removal of solvent under vacuum resulted in an orange to yellow colored oil (m/z 560.6, when the vitamin is α-tocopherol and the amino acid is lysine).

### Example 2

### Preparation of Nano-Particles of an Oligonucleotide and an Amino Acid Ester

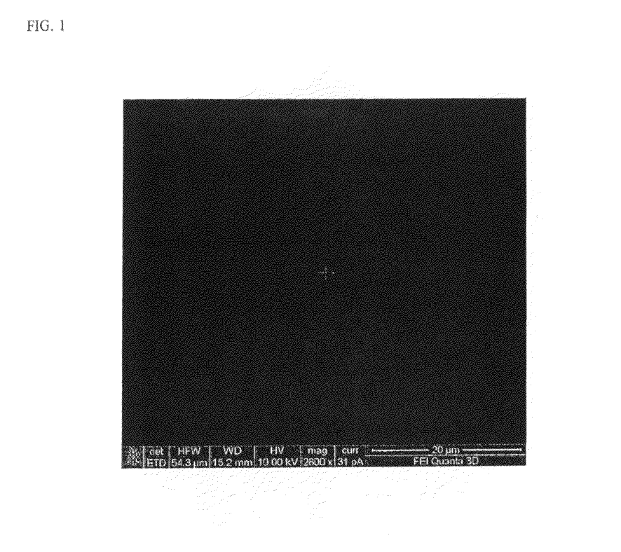

A protonated aptamer of 23 nucleotides was dissolved in dimethylacetamide (80 mg/mL). The aptamer was similar to ARC259, described above, except that the aptamer was pegylated at both the 3′-end and the 5′-end, rather than only at the 5′-end, with a PEG moiety having an average molecular weight of 20 kD. To the resulting solution of the aptamer was added 6 equivalents of phenylalanine hexadecyl ester with stirring. Nano-particles were prepared by adding 10 μL of the dimethylacetamide solution to 1 mL of deionized water and immediately vortexing the resulting composition to provide an aqueous suspension. 50 μL of the resulting suspension was then mixed with 50 μL of Quantomix imaging buffer (commercially available from Electron Microscopy Sciences, Hatfield, Pa.) and 20 μL of the resulting diluted suspension was transferred to Quantaomix QX-102 WET-SEM imaging cell (commercially available from Electron Microscopy Sciences, Hatfield, Pa.). The diluted suspension in the imaging cells was then imaged at North Carolina State University, Dept. of Materials Science and Engineering analytical instrumentation facility (Raleigh, N.C.). The micrograph depicted in FIG. 1 is illustrative of the imaging.

FIG. 1 shows that the particles are in the nano-particle range. It is believed that the nano-particles comprise both the aptamer and the phenylalanine hexadecyl ester.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosure of which are incorporated herein by reference

What is claimed is:

1. A pharmaceutical composition comprising nano-particles or micro-particles comprising: (i) a protonated oligonucleotide and (ii) a pharmaceutically acceptable organic base.

2. The pharmaceutical composition of claim 1, further comprising a solvent, wherein the nano-particles or micro-particles are dispersed in the solvent and the pharmaceutical composition is injectable.

3. The pharmaceutical composition of claim 1, wherein the oligonucleotide is a siRNA.

4. The pharmaceutical composition of claim 1, wherein the oligonucleotide is an aptamer.

5. The pharmaceutical composition of claim 1, wherein the pharmaceutically acceptable organic base is an amino acid ester of formula (I): 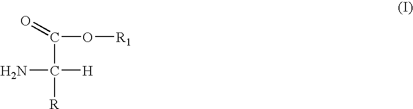
wherein
R is the amino acid side chain; and
R₁ is a C₁ to C₂₂ hydrocarbon group.

6. The pharmaceutical composition of claim 1, wherein the pharmaceutically acceptable organic base is an amino acid amide of formula (II): 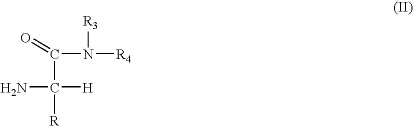
wherein
R is the amino acid side chain;
R₃ is hydrogen or a C₁ to C₂₂ hydrocarbon group; and
R₄ is hydrogen or a C₁ to C₂₂ hydrocarbon group.

7. The pharmaceutical composition of claim 1, wherein the pharmaceutically acceptable organic base is an amino acid-vitamin ester of formula: 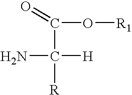
wherein
R is the amino acid side chain; and
O—R₁ is the residue of a vitamin.

8. The pharmaceutical composition of claim 7, wherein O—R₁ is a residue of a vitamin selected from the group consisting of vitamin A, vitamin B₁, vitamin B₂, vitamin B₅, vitamin B₆, vitamin B₁₂, vitamin C, vitamin D, and vitamin E.

9. The pharmaceutical composition of claim 1, wherein the particles are nano-particles.

10. The pharmaceutical composition of claim 1, wherein the particles are micro-particles.

11. A pharmaceutical compositions comprising nano-particles or micro-particles comprising (i) an oligonucleotide, (ii) a divalent metal cation and (iii) optionally a carboxylate, a phospholipid, a phosphatidyl choline, or a sphingomyelin.

12. The pharmaceutical composition of claim 11, wherein the divalent metal cation is selected from the group consisting if alkaline earth metal cations, Mg⁺², Zn⁺², Cu⁺², and Fe⁺².

13. The pharmaceutical composition of claim 11, wherein the carboxylate is a carboxylate derived from an N-acyl amino acid of formula (III): 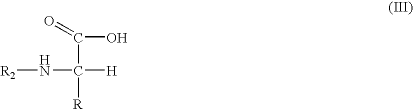
wherein:
R is the amino acid side chain and is defined above; and
R₂ is an acyl group of formula —C(O)—R₅, wherein R₅ is a substituted C₁ to C₂₁ hydrocarbon group.

14. The pharmaceutical composition of claim 11, wherein the particles are nano-particles.

15. The pharmaceutical composition of claim 11, wherein the particles are micro-particles.

16. A method of administering an oligonucleotide to an animal comprising administering to the animal a composition comprising nano-particles or micro-particles comprising: (i) a protonated oligonucleotide and (ii) a pharmaceutically acceptable organic base.

17. The method of claim 12, wherein the nano-particles are dispersed in a solvent and the administering is by injection.

18. The method of claim 16, wherein the particles are nano-particles.

19. The method of claim 16, wherein the particles are micro-particles.

20. A method of administering an oligonucleotide to an animal comprising administering to the animal a composition comprising nano-particles or micro-particles comprising: (i) an oligonucleotide, (ii) a divalent metal cation, and (iii) optionally a carboxylate, a phospholipid, a phosphatidyl choline, or a sphingomyelin.

21. The method of claim 15, wherein the nano-particles are dispersed in a solvent and the administering is by injection.

22. The method of claim 20, wherein the particles are nano-particles.

23. The method of claim 20, wherein the particles are micro-particles.
